(19) United States
(12) Patent Application Publication
(10) Pub. No.: US 2006/0183735 A1
(43) Pub. Date: Aug. 17, 2006

(54) Methods for identifying compounds for regulating muscle mass or function using dopamine receptors

(75) Inventors: Robert Joseph Isfort, Fairfield, OH (US); Russell James Sheldon, Fairfield, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/345,948

(22) Filed: Feb. 2, 2006

Related U.S. Application Data
(62) Division of application No. 10/299,642, filed on Nov. 18, 2002, now Pat. No. 7,067,113.
(60) Provisional application No. 60/349,620, filed on Jan. 18, 2002.

(51) Int. Cl. A61K 31/55; C12Q 1/00

(52) U.S. Cl. 514/217.02; 435/4

(57) ABSTRACT

Screening methods for identifying compounds that bind to or activate (D₁ or D₅ dopamine receptors individually or in combination) or regulate or potentially regulate skeletal muscle mass or function in vivo. Also disclosed are screening methods for identifying compounds that prolong or augment the activation of D₁ or D₅ dopamine receptors or of D₁ or D₅ dopamine receptor signal transduction pathways and increase D₁ or D₅ dopamine receptor expression. Pharmaceutical compositions comprising D₁ or D₅ dopamine receptor agonists, antibodies to D₁ or D₅ dopamine receptors and methods for increasing skeletal muscle mass or function or for the treatment of skeletal muscle atrophy using D₁ or D₅ dopamine receptors as the target for intervention and methods for treatment of muscular dystrophies are described.

### CROSS REFERENCE TO RELATED APPLICATIONS

This application is a divisional application of co-pending U.S. application Ser. No. 10/299,642, filed 18 Nov. 2002, and claims priority under Title 35, United States Code 119(e) from U.S. Provisional Application Ser. No. 60/349,620 filed on Jul. 1, 2002, both of which are herein incorporated by reference in their entirety.

### TECHNICAL FIELD

The present invention relates to methods of identifying candidate compounds for regulating skeletal muscle mass or function or regulating the activity or expression of a dopamine receptor (dopamine receptor). The invention also relates to methods for the treatment of skeletal muscle atrophy or methods for inducing skeletal muscle hypertrophy using D₁ or D₅ dopamine receptors as the target for intervention and to methods of treating muscular dystrophies using D₁ or D₅ dopamine receptors as targets.

### BACKGROUND

### Dopamine Receptors

Dopamine has multiple physiological effects including central and peripheral activities. In the brain, dopamine controls a multitude of functions including locomotor activity, cognition, emotion, positive reinforcement, food intake and endocrine regulation. In the periphery, dopamine functions as a modulator of cardiovascular activity (both cardiac and vascular function), catecholamine release, hormone secretion, renal function and gastrointestinal motility (reviewed in Missale et al., 1998).

Dopamine mediates its action via at least 5 known dopamine receptors (D₁-D₅). These five receptors can be subdivided into two general groups based on their molecular structures, pharmacological activities, and physiological functions as the D₁/D₅ group (D₁-like) and the D_{2/3/4} group (D₂-like) (Civelli et al., 1993; Gingrich et al., 1993; Jackson et al., 1994; Missale et al., 1998; O'Dowd, 1993). The D₁/D₅ subclass of receptors signal predominantly by coupling to Gas, leading to the activation of adenylyl cyclase and the formation of cAMP (Gingrich et al., 1993; Missale et al., 1998). cAMP as a second messenger, has pleotropic effects including the activation of protein kinase A, phospholipase C activation, increase in intracellular calcium and sodium concentrations, changes in intracellular pH, mitogen-activated protein kinase induction, etc (Missale et al., 1998). The D_{2/3/4} subclass of receptors signal mainly by coupling to Gαi, thereby inhibiting the activity of adenylyl cyclase (Gingrich et al., Missale et al., 1998). D₁/D₅ receptor subclass has been observed to also couple to Gαo, Gαi, and Gαq indicating that the signal transduction pathways activated by the G₁/₅ subclass may be quite complex (Kimura et al., 1995a; Sidhu et al., 1991; Wang et al., 1995). Dopamine receptors have been cloned from many species including human (Missale et al., 1998). Expression analysis of the dopamine receptors has demonstrated that the D₁ receptor is expressed widely in the rat brain including the striatum, nucleus accumbens, olfactory tubercle, limbic system, anterior cortex, thalamus, medulla, amygdala, mesencephalon, septum, anterior/posterior basal ganglia, and hypothalamus while the D₅ receptor is expressed in the rat hippocampus, lateral mamillary nucleus, parafascicular nucleus of the thalamus, cerebral cortex, lateral thalamus, substantia nigra, medial thalamus, and hippocampus; in the primate brain the D₁ and D₅ receptors are expressed in pyramidal neurons of prefontal, premotor, cingulate and entorhinal cortex, the hippocampus, the dentate gyrus, olfactory bulb, amygdala, caudate nucleus, and substantia nigra (D₁ only); in the periphery, the D₁/D₅ subbclass of receptors are expressed in blood vessels, adrenal gland, and kidney (Jackson et al., 1994; Missale et al., 1998).

Pharmacologically, agonists that selectively activate and antagonists that selectively block agonist activity of the D₁/D₅ receptor subclass have been described (Missale et al., 1998; Seeman et al., 1994; Sokoloff et al. 1995). These agonist and antagonists are able to differentiate the different dopamine receptors functionally and have been useful in matching biological activity with a specific dopamine receptor class.

Skeletal muscle is a plastic tissue, which readily adapts to changes in either physiological demand for work or metabolic need. Hypertrophy refers to an increase in skeletal muscle mass while skeletal muscle atrophy refers to a decrease in skeletal muscle mass. Acute skeletal muscle atrophy is traceable to a variety of causes including, but not limited to: disuse due to surgery, bed rest, or broken bones; denervation/nerve damage due to spinal cord injury, autoimmune disease, or infectious disease; glucocorticoid use for unrelated conditions; sepsis due to infection or other causes; nutrient limitation due to illness or starvation; and space travel. Skeletal muscle atrophy occurs through normal biological processes, however, in certain medical situations this normal biological process results in a debilitating level of muscle atrophy. For example, acute skeletal muscle atrophy presents a significant limitation in the rehabilitation of patients from immobilizations, including, but not limited to, those accompanying an orthopedic procedure. In such cases, the rehabilitation period required to reverse the skeletal muscle atrophy is often far longer than the period of time required to repair the original injury. Such acute disuse atrophy is a particular problem in the elderly, who may already suffer from substantial age-related deficits in muscle function and mass, because such atrophy can lead to permanent disability and premature mortality.

Skeletal muscle atrophy can also result from chronic conditions such as cancer cachexia, chronic inflammation, AIDS cachexia, chronic obstructive pulmonary disease (COPD), congestive heart failure, genetic disorders, e.g., muscular dystrophies, neurodegenerative diseases and sarcopenia (age associated muscle loss). In these chronic conditions, skeletal muscle atrophy can lead to premature loss of mobility, thereby adding to the disease-related morbidity.

Little is known regarding the molecular processes which control atrophy or hypertrophy of skeletal muscle. While the initiating trigger of the skeletal muscle atrophy is different for the various atrophy initiating events, several common biochemical changes occur in the affected skeletal muscle fiber, including a decrease in protein synthesis and an increase in protein degradation and changes in both contractile and metabolic enzyme protein isozymes characteristic of a slow (highly oxidative metabolism/slow contractile protein isoforms) to fast (highly glycolytic metabolism/fast contractile protein isoforms) fiber switch. Additional changes in skeletal muscle which occur include the loss of vasculature and remodeling of the extracellular matrix. Both fast and slow twitch muscle demonstrate atrophy under the appropriate conditions, with the relative muscle loss depending on the specific atrophy stimuli or condition. Importantly, all these changes are coordinately regulated and are switched on or off depending on changes in physiological and metabolic need.

The processes by which atrophy and hypertrophy occur are conserved across mammalian species. Multiple studies have demonstrated that the same basic molecular, cellular, and physiological processes occur during atrophy in both rodents and humans. Thus, rodent models of skeletal muscle atrophy have been successfully utilized to understand and predict human atrophy responses. For example, atrophy induced by a variety of means in both rodents and humans results in similar changes in muscle anatomy, cross-sectional area, function, fiber type switching, contractile protein expression, and histology. In addition, several agents have been demonstrated to regulate skeletal muscle atrophy in both rodents and in humans. These agents include anabolic steroids, growth hormone, insulin-like growth factor 1, and β-adrenergic agonists. Together, these data demonstrate that skeletal muscle atrophy results from common mechanisms in both rodents and humans.

While some agents have been shown to regulate skeletal muscle atrophy and are approved for use in humans for this indication, these agents have undesirable side effects such as hypertrophy of cardiac muscle, neoplasia, hirsutism, androgenization of females, increased morbidity and mortality, liver damage, hypoglycemia, musculoskeletal pain, increased tissue turgor, tachycardia, and edema. Currently, there are no highly effective and selective treatments for either acute or chronic skeletal muscle atrophy. Thus, there is a need to identify other therapeutic agents which regulate skeletal muscle atrophy.

### Muscular Dystrophies

Muscular dystrophies encompass a group of inherited, progressive muscle disorders, distinguished clinically by the selective distribution of skeletal muscle weakness. The two most common forms of muscle dystrophy are Duchenne and Becker dystrophies, each resulting from the inheritance of a mutation in the dystrophin gene, which is located at the Xp21 locus. Other dystrophies include, but are not limited to, limb-girdle muscular dystrophy which results from mutation of multiple genetic loci including the p94 calpain, adhalin, γ-sarcoglycan, and β-sarcoglycan loci; fascioscapulohumeral (Landouzy-Dejerine) muscular dystrophy, myotonic dystrophy, and Emery-Dreifuss muscular dystrophy. The symptoms of Duchenne muscular dystrophy, which occurs almost exclusively in males, include a waddling gait, toe walking, lordosis, frequent falls and difficulty in standing up and climbing stairs. Symptoms start at about 3-7 years of age with most patients confined to a wheelchair by 10-12 years and many die at about 20 years of age due to respiratory complications. Current treatment for Duchenne muscular dystrophy includes administration of prednisone (a corticosteroid drug), which while not curative, slows the decline of muscle strength and delays disability. Corticosteroids, such as prednisone, are believed to act by blocking the immune cell activation and infiltration which are precipitated by muscle fiber damage resulting from the disease. Unfortunately, corticosteroid treatment also results in skeletal muscle atrophy which negates some of the potential benefit of blocking the immune response in these patients. Thus, there is a need to identify therapeutic agents which slow the muscle fiber damage and delay the onset of disability in patients with muscular dystrophies, but cause a lesser degree of skeletal muscle atrophy than current therapies.

One problem associated with identification of compounds for use in the treatment of skeletal muscle atrophy or of muscular dystrophies has been the lack of good screening methods for the identification of such compounds. Applicants have now found that D₁ and D₅ dopamine receptors are involved in the regulation of skeletal muscle mass or function and that agonists of D₁ and D₅ dopamine receptors are able to block skeletal muscle atrophy and/or induce hypertrophy of skeletal muscle. The present invention solves the problem of identifying compounds for the treatment of muscle atrophy by providing screening methods using D₁ or D₅ dopamine receptors which can be used to identify candidate compounds useful for the treatment of muscle atrophy. The present invention also solves the problem of finding compounds for treatment of muscle dystrophies by providing a screening method to identify candidate compounds which activate both D₁ or D₅ dopamine receptors.

All documents cited are, in relevant part, incorporated herein by reference; the citation of any document is not to be construed as an admission that it is prior art with respect to the present invention.

### SUMMARY OF THE INVENTION

The present invention relates to the use of D₁ or D₅ dopamine receptors to identify candidate compounds that are potentially useful in the treatment of skeletal muscle atrophy and or to induce skeletal muscle hypertrophy. The D₁ and D₅ receptors can be used to identify candidate compounds individually or in combination with each other. In particular, the invention provides in vitro methods for identifying candidate compounds for regulating skeletal muscle mass or function comprising contacting a test compound with a cell expressing D₁ or D₅ dopamine receptors, or contacting a test compound with isolated D₁ or D₅ dopamine receptors, and determining whether the test compound either binds to or activates the D₁ or D₅ dopamine receptors. Another embodiment of the invention relates to a method for identifying candidate therapeutic compounds from a group of one or more candidate compounds which have been determined to bind to or activate D₁ or D₅ dopamine receptors comprising administering the candidate compound to a non-human animal and determining whether the candidate compound regulates skeletal muscle mass or muscle function in the treated animal.

A further embodiment of the invention relates to a method for identifying candidate compounds for regulating skeletal muscle mass or function comprising, in any order: (i) contacting a test compound with a cell expressing a functional D₁ or D₅ dopamine receptor, and determining a level of activation of D₁ or D₅ dopamine receptors resulting from the test compound; (ii) contacting a test compound with a cell expressing a functional D₁ or D₅ dopamine receptor, and determining the level of activation of D₁ or D₅ resulting from the test compound; followed by (iii) comparing the level of D₁ or D₅ dopamine receptor activation and the level of activation; and (iv) identifying those test compounds that show similar activity toward D₁ or D₅ dopamine receptors and or show selectivity for D₁ or D₅ dopamine receptors as candidate compounds for regulating skeletal muscle mass or function.

The invention further provides methods for identifying candidate compounds that prolong or augment the agonist-induced activation of D₁ or D₅ dopamine receptors or of a D₁ or D₅ dopamine receptor signal transduction pathway. These methods comprise in any order or concurrently: (i) contacting a test compound with a cell which expresses functional D₁ or D₅ dopamine receptors; (ii) treating the cell with a D₁ or D₅ dopamine receptors agonist for a sufficient time and at a sufficient concentration to cause desensitization of the D₁ or D₅ dopamine receptors in control cells; followed by (iii) determining the level of activation of D or D₅ dopamine receptors and identifying test compounds that prolong or augment the activation of a dopamine receptor or a dopamine receptor signal transduction pathway as candidate compounds for regulating skeletal muscle mass or function. In a particular embodiment, the present invention relates to a method of identifying candidate therapeutic compounds from a group of one or more candidate compounds determined to prolong or augment the activation of D₁ or D₅ dopamine receptors or activation of D₁ or D₅ dopamine receptors signal transduction pathway comprising: administering the candidate compound, in conjunction with a D₁ or D₅ dopamine receptors agonist, to a non-human animal and determining whether the candidate compound regulates skeletal muscle mass or function in the treated animal.

The invention further provides methods for identifying candidate compounds that increase D₁ or D₅ dopamine receptor expression comprising contacting a test compound with a cell or cell lysate containing a reporter gene operatively associated with a dopamine receptors gene regulatory element and detecting expression of the reporter gene. Test compounds that increase expression of the reporter gene are identified as candidate compounds for increasing D₁ or D₅ dopamine receptor expression. In a particular embodiment, the present invention relates to a method of determining whether those candidate compounds which increase D₁ or D₅ dopamine receptors expression can be used to regulate skeletal muscle mass or function in vivo by administering a candidate compound to a non-human animal and determining whether the candidate compound regulates skeletal muscle mass or function in the treated animal.

The present invention also relates to the use of D₁ or D₅ dopamine receptors agonists, expression vectors encoding a functional D₁ or D₅ dopamine receptor, expression vectors encoding a constitutively active D₁ or D₅ dopamine receptors or compounds that increase expression of D₁ or D₅ dopamine receptors to treat skeletal muscle atrophy. In particular, the invention provides methods of treating skeletal muscle atrophy, in a subject in need of such treatment, comprising administering to the subject a safe and effective amount of a D₁ or D₅ dopamine receptor agonist, an expression vector encoding a functional D₁ or D₅ dopamine receptor, an expression vector encoding a constitutively active D₁ or D₅ dopamine receptor, an expression vector encoding a dopamine receptor or dopamine receptor analog, or a compound that increases expression of D₁ or D₅ dopamine receptors. In a particular embodiment, the present invention relates to a method for treating skeletal muscle atrophy in a subject in need of such treatment comprising administering to the subject a safe and effective amount of a D₁ or D₅ dopamine receptor agonist in conjunction with a safe and effective amount of a compound that prolongs or augments the agonist-induced activation of D₁ or D₅ dopamine receptors, or of a D₁ and D₅ dopamine receptors signal transduction pathway.

The present invention also relates to the use of a D₁ or D₅ dopamine receptors agonist to increase skeletal muscle mass or function in a subject. In particular, the invention provides methods of increasing skeletal muscle mass or function in a subject in which such an increase is desirable, comprising identifying a subject in which an increase in muscle mass or function is desirable and administering to the subject a safe and effective amount of a dopamine agonist.

The invention further provides for pharmaceutical compositions comprising a safe and effective amount of D₁ or D₅ dopamine receptors agonist and a pharmaceutically-acceptable carrier. In a particular embodiment the pharmaceutical composition comprises a chimeric or human antibody specific for D₁ and D₅ dopamine receptors. The present invention also provides for antibodies to D₁ and D₅ dopamine receptors and in particular to chimeric or human antibodies that are agonists of D₁ and D₅ dopamine receptors.

### Sequence Listing Description

Each of the dopamine receptor nucleotide and protein sequences or dopamine receptor analog protein sequence included in the sequence listing, along with the corresponding Genbank or Derwent accession number(s) and animal species from which it is cloned, is shown in Table I. Also shown are accession numbers for related nucleotide sequences that encode identical, or nearly identical, amino acid sequences as the sequence shown in the sequence listing.

TABLE 1

| | | | Genbank (GB) or | |
|---|---|---|---|---|
| | SEQ ID | | Derwent (D) | |
| | NO: | | Accession No. for | Related Genbank |
| Sequence | nucleotide, | | nucleotide | (GB) or Derwent (D) |
| description | amino acid | Species | sequence | Accession Nos. |
| Dopamine | 1, 2 | Homo | X58987 (GB) | AAQ14954 (D) |
| D₁ Receptor | | sapiens | | AAQ43964 (D) |
| Dopamine | 3, 4 | Homo | S58542 (GB) | X55760 (GB) |
| D₁ Receptor | | sapiens | | |
| variant | | | | |
| Dopamine | 5, 6 | Homo | X55758 (GB) | |
| D₁ Receptor | | sapiens | | |
| variant | | | | |
| Dopamine | 7, 8 | Homo | X58454 (GB) | |
| D₅ Receptor | | sapiens | | |
| Dopamine | 9, 10 | Homo | M67439 (GB) | |
| D₅ Receptor | | sapiens | | |
| variant | | | | |
| Dopamine | 11, 12 | Homo | I73473 (GB) | |
| D₅ Receptor | | sapiens | I12852 (GB) | |
| variant | | | | |
| Dopamine | 13, 14 | Homo | M67441 | M77186 (GB) |
| D₅ Receptor | | sapiens | | M67449 (GB) |
| | | pseudogene | | I12853 (GB) |
| | | | | I12854 (GB) |
| | | | | M76064 (GB) |
| | | | | M75867 (GB) |
| | | | | I73474 (GB) |
| | | | | M77185 (GB) |
| | | | | I73473 (GB) |
| | | | | AAT99205 (D) |
| | | | | AAT99204 (D) |
| Dopamine | 15, 16 | Rhesus | AF077862 | |
| D₁ Receptor | | macaque | | |
| Dopamine | 17, 18 | Gorilla | S77846 | |
| D₅ Receptor | | gorilla | | |
| Dopamine | 19, 20 | Rattus | S46131 | AAQ14955 (D) |
| D₁ Receptor | | norvegicus | | |
| Dopamine | 21, 22 | Rattus | M35077 (GB) | |
| D₁ Receptor | | norvegicus | I58000 (GB) | |
| variant | | | | |
| Dopamine | 23, 24 | Rattus | M69118 | |
| D₅ Receptor | | norvegicus | | |
| Dopamine | 25, 26 | Gallus | L36877 | |
| D₁ Receptor | | domesticus | | |
| Dopamine | 27, 28 | Anguilla | U62918 | |
| D₁ Receptor | | anguilla | | |
| Dopamine | 29, 30 | Didelphis | S67258 | |
| D₁ Receptor | | virginiana | | |
| Dopamine | 31, 32 | Sus scrofa | U25681 | |
| D₁ Receptor | | | | |

### BRIEF DESCRIPTION OF THE FIGURES

FIGS. 1A and 1B demonstrates the anti-atrophy effect of the D₁ and D₅ dopamine receptor agonists, SKF 81297 (administered subcutaneously, 2× daily), on the tibialis anterior (FIG. 1A) and medial gastrocnemius (FIG. 1B) muscles in the mouse sciatic nerve denervation atrophy model. FIGS. 1A and 1B also demonstrate the hypertrophy inducing effect of SKF 81297 on the non-denervated (normal) tibialis anterior (FIG. 1A) and medial gastrocnemius (FIG. 1B) muscles.

FIG. 2 demonstrates the anti-atrophy effect of the D₁ and D₅ dopamine receptor agonists, SKF 81297 (administered subcutaneously, 2× daily), on casting-induced atrophy of the tibialis anterior muscle and the hypertrophy inducing effect of SKF 81297 on the non-casted (normal) tibialis anterior muscle.

FIG. 3 demonstrates the anti-atrophy effect of the D₁ and D₅ dopamine receptor agonists, fenoldopam (administered subcutaneously, 2× daily), on casting-induced atrophy of the medial gastrocnemius muscle.

### DETAILED DESCRIPTION OF THE INVENTION

I. Terms and Definitions:

The following is a list of definitions for terms used herein.

“Agonist” means any compound, including, but not limited to, antibodies, that activates a receptor. For example, dopamine receptor agonists include, but are not limited to, dopamine and dopamine analogs; for example SKF81297 and Fenoldopam.

“Allelic variant” means a variant form of a given gene or gene product. One of skill in the art recognizes that a large number of genes are present in two or more allelic forms in a population and some genes have numerous alleles.

“Binding affinity” means the propensity for a ligand to interact with a receptor and is inversely related to the dissociation constant for a specific dopamine receptor ligand-dopamine receptor interaction. The dissociation constant can be measured directly via standard saturation, competition, or kinetics binding techniques or indirectly via pharmacological techniques involving functional assays and endpoints.

“Chimeric antibody” means an antibody that contains structural elements from two or more different antibody molecules, i.e., from different animal species. Chimeric antibodies include, but are not limited to, antibodies known as “humanized antibodies” which include, but are not limited to, chimeric antibodies generated by the technique known as complementarity determining region grafting.

“Dopamine receptor agonist” means a compound or molecule which has the ability to activate D₁ or D₅ dopamine receptors, or both. Activation of dopamine receptors can be measured as described hereinafter; using selective agonist such as, SKF 81297 and Fenoldopam D₁/D₅ selective receptor agonists.

“Dopamine receptor” means D₁ or D₅ dopamine receptor from any biological species.

The term “dopamine receptor” also includes truncated and/or mutated proteins wherein regions of the receptor molecule not required for ligand binding or signaling have been deleted or modified. For example, one of skill in the art will recognize that a dopamine receptor with one or more conservative changes in the primary amino acid sequence would be useful in the present invention. It is known in the art that substitution of certain amino acids with different amino acids with similar structure or properties (conservative substitutions) can result in a silent change, i.e., a change that does not significantly alter function. Conservative substitutes are well known in the art. For example, it is known that GPCRs can tolerate substitutions of amino acid residues in the transmembrane alpha-helices, which are oriented toward lipid, with other hydrophobic amino acids, and remain functional. D₁ and D₅ dopamine receptors differing from a naturally occurring sequence by truncations and/or mutations such as conservative amino acid substitutions are also included in the definition of dopamine receptors.

One of skill in the art would also recognize that dopamine receptors from a species other than those listed above, particularly mammalian species, would be useful in the present invention. One of skill in the art would further recognize that by using probes from the known dopamine receptor species' sequences, cDNA or genomic sequences homologous to the known sequence could be obtained from the same or alternate species by known cloning methods. Such are also included in the definition of and such D₁ and D₅ dopamine receptors are also included in the definition of D₁ and D₅ dopamine receptors.

In addition, one of skill in the art would recognize that functional allelic variants or functional splice variants of dopamine receptors might be present in a particular species and that these variants would have utility in the present invention. Such variants are also included in the definition of and such D₁ and D₅ dopamine receptors variants are also included in the definition of D₁ and D₅ dopamine receptors.

Fusions of D₁ and D₅ dopamine receptors polypeptide, or D₁ and D₅ dopamine receptors polypeptide fragment to a non-dopamine receptor polypeptide are referred to as dopamine receptor fusion proteins. Using known methods, one of skill in the art would be able to make fusion proteins of a D₁ and D₅ dopamine receptors that, while different from native and D₁ and D₅ dopamine receptors, would remain useful in the present invention. For example the non-dopamine receptor polypeptide may be a signal (or leader) polypeptide sequence which co-translationally or post-translationally directs transfer of the protein from its site of synthesis to another site (e.g., the yeast α-factor leader). Or the non-dopamine receptor polypeptide may be added to facilitate purification or identification of the dopamine receptor (e.g., poly-His, or Flag peptide). D₁ and D₅ dopamine receptors fusion proteins are also included within the definition of D₁ and D₅ dopamine receptors.

“D₁ and D₅ dopamine receptors signal transduction pathway” means any signaling pathway (e.g., cAMP, MAP kinase) or combination of signaling pathways that are modulated by the binding of endogenous or exogenous ligands to D₁ and D₅ dopamine receptors.

“Fenoldopam” is 6-chloro-2, 3, 4, 5-tetrahydro-1-(4-hydroxyphenyl)-[1H]-3-benzazepine-7,8-diol, also known as Corlopam®

“Functional dopamine receptors” refers to dopamine receptors, which bind dopamine receptor agonists in vivo or in vitro and are activated as a result of ligand binding.

“Fusion gene” means two or more DNA coding sequences operably associated so as to encode one hybrid protein. A “fusion protein” is the protein product of a fusion gene.

“Inhibit” means to partially or completely block a particular process or activity. For example, a compound inhibits skeletal muscle atrophy if it either completely or partially prevents muscle atrophy.

“Operably associated” refers two DNA sequences where the nature of the linkage between the two DNA sequences does not (1) result in the introduction of a frame-shift mutation, (2) interfere with the ability of a promoter region to direct the transcription of the coding sequences, or (3) interfere with the ability of the corresponding RNA transcript to be translated into a protein. For example, a coding sequence and regulatory sequences are operably associated when they are covalently linked in such a way as to place the transcription of the coding sequence under the influence or control of the regulatory sequences. Thus, a promoter region is operably associated with a coding sequence when the promoter region is capable of effecting transcription of that DNA sequence such that the resulting transcript is capable of being translated into the desired protein or polypeptide.

“Percent identity” means the percentage of nucleotides or amino acids that two sequences have in common, calculated as follows. To calculate the percent identity for a specific sequence (the query), the relevant part of the query sequence is compared to a reference sequence using the BestFit comparison computer program, Wisconsin Package, Version 10.1, available from the Genetics Computer Group, Inc. This program uses the algorithm of Smith and Waterman, Advances in Applied Mathematics, Issue 2: 482-489 (1981). Percent identity is calculated with the following default parameters for the BestFit program: the scoring matrix is blosum62.cmp, the gap creation penalty is 8 and the gap extension penalty is 2. When comparing a sequence to the reference sequence, the relevant part of the query sequence is that which is derived from a dopamine receptor sequence. For example, where the query is a dopamine receptor/purification tag fusion protein, only the dopamine receptor polypeptide portion of the sequence is aligned to calculate the percent identity score.

“Polypeptide” means any chain of amino acids, regardless of length or post-translational modification (e.g., phosphorylation or glycosylation).

“Promoter” means a DNA sequence which controls the initiation of transcription and the rate of transcription from a gene or coding region.

“Prophylactic treatment” means preventive treatment of a subject, not currently exhibiting signs of skeletal muscle atrophy, in order to completely or partially block the occurrence of skeletal muscle atrophy. One of skill in the art would recognize that certain individuals are at risk for skeletal muscle atrophy as discussed in the background section herein. Furthermore, one of skill in the art would recognize that if the biochemical changes leading to skeletal muscle atrophy are appropriately regulated, that the occurrence of atrophy would be prevented or reduced in at-risk individuals. For example, muscular dystrophy patients beginning treatment with corticosteroids are at risk for developing skeletal muscle atrophy indicating that prophylactic treatment of such patients would be appropriate.

“Regulate” in all its grammatical forms, means to increase, decrease or maintain, e.g., to regulate skeletal muscle mass or function means to increase, decrease or maintain the level of skeletal muscle mass or function.

“Regulation of skeletal muscle mass or function” includes regulation of skeletal muscle mass, skeletal muscle function or both.

“Regulatory element” means a DNA sequence that is capable of controlling the level of transcription from an operably associated DNA sequence. Included within this definition of regulatory element are promoters and enhancers. E.g., a dopamine receptor gene regulatory element is a DNA sequence capable of controlling the level of transcription from the dopamine receptor gene.

“Reporter gene” means a coding sequence whose product can be detected, preferably quantitatively, wherein the reporter gene is operably associated with a heterologous promoter or enhancer element which is responsive to a signal which is to be measured. The promoter or enhancer element in this context is referred to herein as a “responsive element”.

“SKF 81297” is 6-chloro-7,8-dihydroxyl-1-phenyl-2, 3, 4, 5-tetrahydro-1H-3-benzazepine purchased from RBI/Sigma, Natick, Mass.

“Selective agonist” means that the agonist has significantly greater activity toward a certain receptor(s) compared with other receptors, not that it is completely inactive with regard to other receptors. For example, D₁ and D₅ dopamine receptor selective agonists are not limited to SKF 81297 and Fenoldopam.

“SKF” means SmithKline French

“Skeletal muscle hypertrophy” means an increase in skeletal muscle mass or skeletal muscle function or both.

“Skeletal muscle atrophy” means the same as “muscle wasting” and means a decrease in skeletal muscle mass or skeletal muscle function or both.

“Splice variant” means an mRNA or protein which results from alternative exon usage. One of skill in the art recognizes that, depending on cell type, or even within a single cell type, an mRNA may be expressed in a different form, as a splice variant, and thus the translated protein will be different depending upon the mRNA that is expressed.

A “therapeutically effective amount” of a substance is an amount capable of producing a medically desirable result in a treated patient, e.g., decreases skeletal muscle atrophy, increases skeletal muscle mass or increases skeletal muscle function, with an acceptable benefit: risk ratio; in a human or non-human mammal.

“Therapeutic treatment” means treatment of a subject in which an increase in muscle mass or muscle function is desirable. For example, treatment of a subject currently exhibiting signs of skeletal muscle atrophy in order to partially or completely reverse the skeletal muscle atrophy that has occurred or to completely or partially block the occurrence of further skeletal muscle atrophy would be therapeutic treatment of that subject. The term “therapeutic treatment” also includes, for example, treatment of a subject not exhibiting signs of skeletal muscle atrophy to induce skeletal muscle hypertrophy, e.g., treatment of a livestock animal to increase muscle mass.

The term “treatment” means prophylactic or therapeutic treatment.

Unless otherwise defined, all technical and scientific terms used herein have the same meaning as commonly understood by one of ordinary skill in the arts of protein chemistry, pharmacology, or molecular biology. The methods, materials and examples described herein are not intended to be limiting. Other methods and materials similar or equivalent to those described herein can be used in the practice or testing of the present invention.

II. The Role of Dopamine Receptors in Regulation of Skeletal Muscle Mass

One of skill in the art would recognize the utility of the present invention given the information in the prior art and the teachings below. The results described herein demonstrate that administration of a dopamine receptor agonist which activates both D₁ and D₅ dopamine receptors (selective dopamine receptor agonist) but not the D_{2/3/4} dopamine receptors blocks and/or inhibits the skeletal muscle atrophy inducing effect of denervation and disuse treatment in models of skeletal muscle atrophy. Furthermore, results demonstrate that administration of selective dopamine receptor agonist show a hypertrophy inducing effect. Together, these data demonstrate the modulatory role of the D₁ and D₅ dopamine receptors in the process of skeletal muscle atrophy. The specific role of dopamine receptors in vivo was investigated using the pharmacological agents, SKF81297 (RBI/Sigma, Natick, Mass.) and Fenoldopam (Corlopam®), which are selective agonists for D₁ and D₅ dopamine receptors in various models of skeletal muscle atrophy, described hereinafter. These agents have been well characterized and are described in the scientific literature.

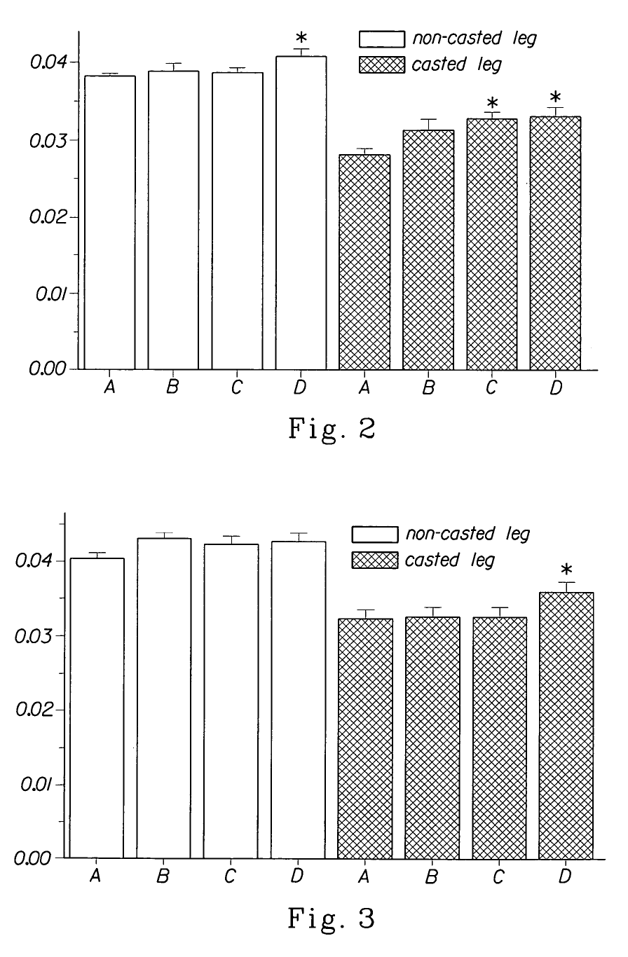

FIG. 2 demonstrates the anti-atrophy effect of the D₁ and D₅ dopamine receptor agonists, SKF 81297 (administered subcutaneously, 2× daily), on casting-induced atrophy of the tibialis anterior muscle and the hypertrophy inducing effect of SKF 81297 on the non-casted (normal) tibialis anterior muscle.

FIG. 3 demonstrates the anti-atrophy effect of the D₁ and D₅ dopamine receptor agonists, fenoldopam (administered subcutaneously, 2× daily), on casting-induced atrophy of the medial gastrocnemius muscle.

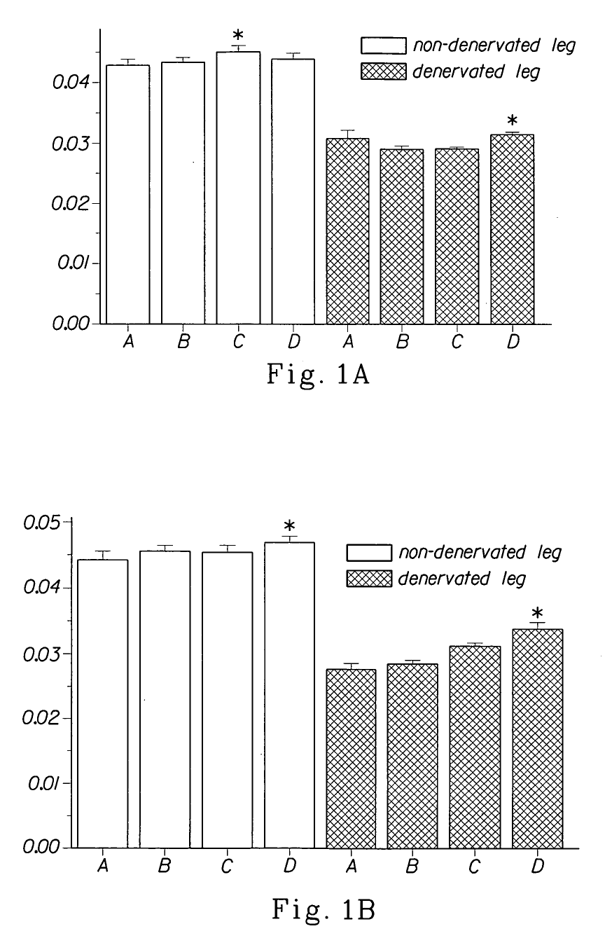

Specifically, FIG. 1 (FIG. 1.) shows that SKF 81297 inhibits denervation-induced atrophy of the tibialis anterior (FIG. 1A) medial gastrocnemius (FIG. 1B) muscles in a mouse sciatic nerve denervation atrophy model. In addition, FIG. 1 demonstrates that SKF 81297 induces hypertrophy in the normal (non-denervated) tibialis anterior (FIG. 1A) and medial gastrocnemius (FIG. 1B) muscles. Legend: A—physiological saline (control); B—SKF 81297 (0.3 mg/kg)+theophylline; C—SKF 81297 (1.0 mg/kg)+theophylline; D—SKF 81297 (3.0 mg/kg)+theophylline. *—p≦0.05 compared to saline. Following denervation of the right sciatic nerve, male mice were injected subcutaneously in the midscapular region twice daily with SKF 81297, at the doses indicated above or vehicle control (physiological saline) for nine days. SKF 81297 was co-administered with twice daily intra-peritoneal dosing of the phosphodiesterase inhibitor theophylline (30 mg/kg). On day nine, the tibialis anterior and medial gastrocnemius muscles were removed and weighed to determine the degree of atrophy.

FIG. 2 (FIG. 2.) demonstrates that SKF 81297 inhibits disuse-induced atrophy of the tibialis anterior muscle. In addition, statistically significant hypertrophy of the tibialis anterior muscles of the non-casted leg was also observed with SKF 81297 treatment. Legend: A—physiological saline (control); B—SKF 81297 (0.3 mg/kg)+theophylline; C—SKF 81297 (1.0 mg/kg)+theophylline; D—SKF 81297 (3.0 mg/kg)+theophylline; *—p≦0.05 compared to saline. Following casting of the right hind leg, male mice were injected subcutaneously in the midscapular region twice daily, with SKF 81297 or vehicle control (physiological saline) for ten days at the daily delivered dose indicated. SKF 81297 was co-administered with twice daily intra-peritoneal dosing of the phosphodiesterase inhibitor theophylline (30 mg/kg). On day ten, the tibialis anterior muscle was removed and weighed to determine the degree of atrophy.

FIG. 3 (FIG. 3.) demonstrates that fenoldopam inhibits disuse-induced atrophy of the medial gastrocnemius muscle. Legend: A—physiological saline (control); B—fenoldopam (0.3 mg/kg); C—fenoldopam (1.0 mg/kg); D—fenoldopam (3.0 mg/kg); *—p≦0.05 compared to saline. Following casting of the right hind leg, male mice were injected subcutaneously in the midscapular region twice daily, with fenoldopam or vehicle control (physiological saline) for ten days at the daily delivered dose indicated. On day ten, the medial gastrocnemius muscle was removed and weighed to determine the degree of atrophy.

III. Preparation of Dopamine Receptors, Dopamine Receptor or Dopamine Receptor Analogs, or Cell Lines Expressing Dopamine Receptors

D₁ and D₅ dopamine receptors can be prepared for a variety of uses, including, but not limited to, the generation of antibodies, use as reagents in the screening assays of the present invention, and use as pharmaceutical reagents for the treatment of skeletal muscle atrophy. It will be clear to one of skill in the art that, for certain embodiments of the invention, purified polypeptides will be most useful, while for other embodiments cell lines expressing the polypeptides will be most useful. For example, in situations where it is important to retain the structural and functional characteristics of the dopamine receptor, e.g., in a screening method to identify candidate compounds which activate dopamine receptors, it is desirable to use cells which express functional dopamine receptors.

Where the source of dopamine receptors is a cell line expressing the polypeptide, the cells may, for example, endogenously express dopamine receptor, have been stimulated to increase endogenous dopamine receptor expression or have been genetically engineered to express a dopamine receptor. Methods for determining whether a cell line expresses a polypeptide of interest are known in the art, for example, detection of the polypeptide with an appropriate antibody, use of a DNA probe to detect mRNA encoding the protein (e.g., northern blot or PCR techniques), or measuring binding of an agent selective for the polypeptide of interest (e.g., a radiolabeled selective agonist).

The use of recombinant DNA technology in the preparation of D₁ and D₅ dopamine receptors, or of cell lines expressing these polypeptides is particularly contemplated. Such recombinant methods are well known in the art. To express recombinant D₁ and D₅ dopamine receptors, an expression vector that comprises a nucleic acid which encodes the polypeptide of interest under the control of one or more regulatory elements, is prepared. Genomic or cDNA sequences encoding and D₁ and D₅ dopamine receptors from several species have been described and are readily available from the GenBank database (available at <http://www.ncbi.nlm.nih.gov/>) or Derwent database (available at <http://www.derwent.co.uk/geneseq/index.html>) as well as in the sequence listing for this application. The accession numbers for and D₁ and D₅ dopamine receptors sequences and corresponding SEQ ID NOS. are shown in Table I. Using this publicly available sequence information, one means of isolating a nucleic acid molecule encoding a D₁ and D₅ dopamine receptor is to screen a genomic DNA or cDNA library with a natural or artificially synthesized DNA probe, using methods well known in the art, e.g., by PCR amplification of the sequence from an appropriate library. Another method is to use oligonucleotide primers specific for the receptor of interest to PCR amplify the cDNA directly from mRNA isolated from a particular tissue (such as skeletal muscle). Such isolated mRNA is commercially available. One of skill in the art would also recognize that by using nucleic acid probes corresponding to portions of the known dopamine receptor sequences the homologous cDNAs or genomic sequences from other species can be obtained using known methods. Particularly useful in the methods of the present invention are dopamine receptors from the species including, but not limited to, human, mouse, rat, pig, monkey, chimpanzee, marmoset, dog, cow, sheep, cat, chicken and turkey. By methods well known in the art, the isolated nucleic acid molecule encoding the dopamine receptor of interest is then ligated into a suitable expression vector. The expression vector, thus prepared, is expressed in a host cell and the host cells expressing the receptor are used directly in a screening assay or the receptor is isolated from the host cells expressing the receptor and the isolated receptor is used in a screening assay.

The host-expression vector systems that may be used for purposes of the invention include, but are not limited to: microorganisms such as bacteria (e.g., E. coli, B. subtilis) transformed with recombinant bacteriophage DNA, plasmid DNA, or cosmid DNA expression vectors containing dopamine receptor nucleotide sequences; yeast (e.g., Saccharomyces, Pichia) transformed with recombinant yeast expression vectors containing dopamine receptor nucleotide sequences; insect cell systems infected with recombinant virus expression vectors (e.g., baculovirus) containing dopamine receptor nucleotide sequences; plant cell systems infected with recombinant virus expression vectors (e.g., cauliflower mosaic virus, tobacco mosaic virus) or transformed with recombinant plasmid expression vectors (e.g., Ti plasmid) containing dopamine receptor nucleotide sequences; or mammalian cell systems (e.g., COS, CHO, HEK293, NIH3T3) harboring recombinant expression constructs containing promoters derived from the genome of mammalian cells (e.g., metallothionein promoter) or from mammalian viruses (e.g., retrovirus LTR) and also containing dopamine receptor nucleotide sequences.

The host cell is used to produce the polypeptide of interest. Because the dopamine receptor is a membrane bound molecule, it is purified from the host cell membranes or the dopamine receptor is utilized while anchored in the cell membrane, i.e., whole cells or membrane fractions of cells are used. Purification or enrichment of the dopamine receptors from such expression systems is accomplished using appropriate detergents and lipid micelles by methods well known to those skilled in the art.

In bacterial systems, a number of expression vectors may be advantageously selected depending upon the use intended for the gene product being expressed. For example, when a large quantity of such protein is produced for the generation of antibodies to dopamine receptors, vectors which direct the expression of high levels of protein products are desirable. One skilled in the art is able to generate such vector constructs and purify the proteins by a variety of methodologies including selective purification technologies such as fusion protein selective columns and antibody columns, and non-selective purification technologies.

In an insect protein expression system, the baculovirus A. californica nuclear polyhedrosis virus (AcNPV), is used as a vector to express foreign genes in S. frugiperda cells. In this case, dopamine receptor nucleotide sequences are cloned into non-essential regions of the virus and placed under the control of an AcNPV promoter. The recombinant viruses are then used to infect cells in which the inserted gene is expressed and the protein is purified by one of many techniques known to one skilled in the art.

In mammalian host cells, a number of viral-based expression systems may be utilized. Utilization of these expression systems often requires the creation of specific initiation signals in the vectors for efficient translation of the inserted nucleotide sequences. This is particularly important if a portion of the dopamine receptor gene is used which does not contain the endogenous initiation signal. The placement of this initiation signal, in frame with the coding region of the inserted nucleotide sequence, as well as the addition of transcription and translation enhancing elements and the purification of the recombinant protein, are achieved by one of many methodologies known to one skilled in the art. Also important in mammalian host cells is the selection of an appropriate cell type which is capable of the necessary post translational modifications of the recombinant protein. Such modifications, for example, cleavage, phosphorylation, glycosylation, etc., require the selection of the appropriate host cell which contains the modifying enzymes. Such host cells include, but are not limited to, CHO, HEK293, NIH3T3, COS, etc. and are known by those skilled in the art.

For long term, high expression of recombinant proteins, stable expression is preferred. For example, cell lines that stably express dopamine receptors may be engineered. One of skill in the art, following known methods such as electroporation, calcium phosphate transfection, or liposome-mediated transfection, can generate a cell line that stably expresses dopamine receptors. This is usually accomplished by transfecting cells using expression vectors which contain appropriate expression control elements (e.g., promoter sequences, enhancer sequences, transcriptional termination sequences, polyadenylation sites, translational start sites, etc.), a selectable marker, and the gene of interest. The selectable marker may either be contained within the same vector, as the gene of interest, or on a separate vector, which is co-transfected with the dopamine receptor sequence containing vector. The selectable marker in the expression vector may confer resistance to the selection and allows cells to stably integrate the vector into their chromosomes and to grow to form foci which in turn can be cloned and expanded into cell lines. Alternatively, the expression vector may allow selection of the cell expressing the selectable marker utilizing a physical attribute of the marker, i.e., expression of Green Fluorescent Protein (GFP) allows for selection of cells expressing the marker using fluorescence activated cell sorting (FACS) analysis.

One of skill in the art is able to select an appropriate cell type for transfection in order to allow for selection of cells into which the gene of interest has been successfully integrated. For example, where the selectable marker is herpes simplex virus thymidine kinase, hypoxanthine-guanine phosphoribosyltransferase or adenine phosphoribosyltransferase, the appropriate cell type would be tk-, hgprt- or aprt-cells, respectively. Or, normal cells can be used where the selectable marker is dhfr, gpt, neo or hygro which confer resistance to methotrexate, mycophenolic acid, G-418 or hygromycin, respectively. Such recombinant cell lines are useful for identification of candidate compounds that affect the dopamine receptor activity.

IV. Preparation of Dopamine Receptor Antibodies

Antibodies that selectively recognize one or more epitopes of a dopamine receptor are also encompassed by the invention. Such antibodies include, e.g., polyclonal antibodies, monoclonal antibodies, chimeric antibodies, human antibodies, single chain antibodies, Fab fragments, F(ab′)₂ fragments, molecules produced using a Fab expression library, human antibodies (polyclonal or monoclonal) produced in transgenic mice and epitope binding fragments of any of the above. For therapeutic uses, chimeric or human antibodies are preferred; human antibodies are most preferred.

The antibodies can be utilized in conjunction with the compound screening schemes described herein for the evaluation of test compounds, e.g., for immobilization of dopamine receptor polypeptides or such antibodies can be used in conjunction with gene therapy techniques to evaluate, for example, the expression of dopamine receptors either in cells or directly in patient tissues in which these genes have been introduced. In addition, antibodies of the present invention are useful in the treatment of skeletal muscle atrophy. Antibodies selective for the dopamine receptor can be screened by the methods of the present invention to identify a subset of the antibodies that are dopamine receptor agonists. In addition, anti-idiotype antibodies generated against antibodies specific for dopamine receptor may be useful as dopamine receptor agonists and like anti-dopamine receptor antibodies may be screened for their ability to activate the dopamine receptor by methods of the present invention.

For the production of antibodies, a variety of host animals may be immunized by injection with dopamine receptors, anti-dopamine receptor antibody, or immunogenic fragments thereof by methods well known in the art. For preparation of an anti-idiotype antibody the immunogen is an anti-dopamine receptor antibody. Production of anti-idiotype antibodies is described, for example, in U.S. Pat. No. 4,699,880, incorporated herein by reference. Suitable host animals include, but are not limited to, rabbits, mice, goats, sheep and horses. Immunization techniques are well known in the art. Polyclonal antibodies can be purified from the serum of the immunized animals, or monoclonal antibodies can be generated by methods that are well known in the art. These techniques include, but are not limited to, the well-known hybridoma techniques of Kohler and Milstein, human B-cell hybridoma techniques, and the EBV hybridoma technology. Monoclonal antibodies may be of any immunoglobulin class, including IgG, IgE, IgM, IgA, and IgD containing either kappa or lambda light chains.

Because of the immunogenicity of non-human antibodies in humans, chimeric antibodies are preferred to non-human antibodies when used for therapeutic treatment of human patients. Techniques of producing and using chimeric antibodies are known in the art, and are described in, for example, U.S. Pat. Nos. 5,807,715; 4,816,397; 4,816,567; 5,530,101; 5,585,089; 5,693,761; 5,693,762; 6,180,370; and 5,824,307, all incorporated herein by reference.

Completely human antibodies are particularly desirable for therapeutic treatment of human patients because they are less immunogenic than non-human antibodies or chimeric antibodies. Such antibodies can be produced using transgenic mice which are substantially incapable of expressing endogenous immunoglobulin heavy and light chain genes, but which can express human heavy and light chain genes. The transgenic mice are immunized in the normal fashion with a selected antigen, e.g., all or a portion of D₁ and D₅ dopamine receptors. Monoclonal antibodies directed against the antigen are obtained using conventional hybridoma technology from these immunized transgenic mice. This technology is described in detail in U.S. Pat. Nos. 5,874,299; 5,877,397; 5,569,825; 5,661,016; 5,770,429; and 6,075,181, all incorporated herein by reference. As an alternative to obtaining human immunoglobulins directly from the culture of the hybridoma cells, the hybridoma cells can be used as a source of rearranged heavy chain and light chain loci for subsequent expression or genetic manipulation. Isolation of genes from such antibody-producing cells is straightforward since high levels of the appropriate mRNAs are available. The recovered rearranged loci can be manipulated as desired. For example, the constant region can be eliminated or exchanged for that of a different isotype or the variable regions can be linked to encode single chain Fv regions. Such techniques are described in WO 96/33735 and WO 96/34096, all incorporated herein by reference.

V. Selection of Test Compounds

Compounds that can be screened in accordance with the assays of the invention include but are not limited to, libraries of known compounds, including natural products, such as plant or animal extracts, synthetic chemicals, biologically active materials including proteins, peptides such as soluble peptides, including but not limited to members of random peptide libraries and combinatorial chemistry derived molecular library made of D- or L-configuration amino acids, phosphopeptides (including, but not limited to, members of random or partially degenerate, directed phosphopeptide libraries), antibodies (including, but not limited to, polyclonal, monoclonal, chimeric, human, anti-idiotypic or single chain antibodies, and Fab, F(ab′)₂ and Fab expression library fragments, and epitope-binding fragments thereof), organic and inorganic molecules.

In addition to the more traditional sources of test compounds, computer modeling and searching technologies permit the rational selection of test compounds by utilizing structural information from the ligand binding site of dopamine receptor or from already identified agonists of dopamine receptors. Such rational selection of test compounds can decrease the number of test compounds that must be screened in order to identify a candidate therapeutic compound. Dopamine receptors are GPCRs, and thus knowledge of the dopamine receptor protein sequence allows for the generation of a model of its binding site that can be used to screen for potential ligands. This process can be accomplished in several manners well known in the art. Briefly, the most robust approach involves generating a sequence alignment of the dopamine receptor sequence to a template (derived from the bacterio-rhodopsin or rhodopsin crystal structures or other GPCR model), conversion of the amino acid structures and refining the model by molecular mechanics and visual examination. If a strong sequence alignment cannot be obtained, then a model may also be generated by building models of the hydrophobic helices. These are then fitted together by rotating and translating each helix relative to the others starting from the general layout of the known rhodopsin structures. Mutational data that point towards residue-residue contacts may also be used to position the helices relative to each other so that these contacts are achieved. During this process, docking of the known ligands into the binding site cavity within the helices may also be used to help position the helices by developing interactions that would stabilize the binding of the ligand. The model may be completed by refinement using molecular mechanics and loop building of the intracellular and extracellular loops using standard homology modeling techniques. General information regarding GPCR structure and modeling can be found in Schoneberg, T. et. al., Molecular and Cellular Endocrinology, 151:181-193 (1999), Flower, D., Biochimica et Biophysica Acta, 1422:207-234 (1999), and Sexton, P. M., Current Opinion in Drug Discovery and Development, 2(5):440-448 (1999).

Once the model is completed, it can be used in conjunction with one of several existing computer programs to narrow the number of compounds to be screened by the screening methods of the present invention. The most general of these is the DOCK program (UCSF Molecular Design Institute, 533 Parnassus Ave, U-64, Box 0446, San Francisco, Calif. 94143-0446). In several of its variants it can screen databases of commercial and/or proprietary compounds for steric fit and rough electrostatic complementarity to the binding site. It has frequently been found that molecules that score well within DOCK have a better chance of being ligands. Another program that can be used is FLEXX (Tripos Inc., 1699 South Hanley Rd., St. Louis, Mo., 63144-2913 (www.tripos.com)). This program, being significantly slower, is usually restricted to searches through smaller databases of compounds. The scoring scheme within FLEXX is more detailed and usually gives a better estimate of binding ability than does DOCK. FLEXX is best used to confirm DOCK suggestions, or to examine libraries of compounds that are generated combinatorially from known ligands or templates.

VI. Screening Assays to Identify Candidate Compounds for the Regulation of Skeletal Muscle Mass or Function

The finding that D₁ and D₅ dopamine receptors play a role in regulating skeletal muscle atrophy hypertrophy enables various methods of screening one or more test compounds to identify candidate compounds that ultimately may be used for prophylactic or therapeutic treatment of skeletal muscle atrophy. This invention provides methods for screening test compounds for their ability to bind to D₁ and D₅ dopamine receptors, activate D₁ and D₅ dopamine receptors, prolong or augment the agonist-induced activation of D₁ and D₅ dopamine receptors or of a D₁ and D₅ dopamine receptor signal transduction pathway or increase expression of D₁ and D₅ dopamine receptor genes.

For screening for compounds which ultimately will be used to regulate skeletal muscle mass or function through D₁ or D₅ dopamine receptors in humans, it is preferred that the initial in vitro screen be carried out using a D₁ dopamine receptor with an amino acid sequence that is greater than 70% identical to SEQ ID NO:2 and more preferably greater than 80% identical to SEQ ID NO:2 and most preferably greater than 90% identical to SEQ ID NO: 2. It is preferred that the initial in vitro screen be carried out using a D₅ dopamine receptor with an amino acid sequence that is greater than 70% identical to SEQ ID NO: 8 and more preferably greater than 80% identical to SEQ ID NO: 8 and most preferably greater than 90% identical to SEQ ID NO: 8. More preferably, the test compounds will be screened against human, mouse or rat dopamine receptors, with the most preferable being human. For screening for compounds which ultimately will be used to regulate skeletal muscle mass or function through D₁ and D₅ dopamine receptors in a non-human species it is preferable to use the D₁ and D₅ dopamine receptors from the species in which treatment is contemplated.

For screening, to determine the level of activity that a test or candidate compound has towards determining what, if any, selectivity a candidate compound exhibits for D₁ and D₅ dopamine receptors, it is preferred that the initial screen be carried out using a D₁ dopamine receptor with an amino acid sequence that is greater than 70% identical to SEQ ID NO:2 and more preferably greater than 80% identical to SEQ ID NO:2 and most preferably greater than 90% identical to SEQ ID NO:2. It is also preferred that the next initial screen be carried out using a D₅ dopamine receptor with an amino acid sequence that is greater than 70% identical to SEQ ID NO: 8 and more preferably greater than 80% identical to SEQ ID NO: 8 and most preferably greater than 90% identical to SEQ ID NO: 8. More preferably the test compounds will be screened against a human, mouse or rat, with the most preferable being human. For screening for compounds which ultimately will be used to regulate skeletal muscle mass or function in a non-human species, it is preferable to use receptors from the species in which treatment is contemplated such as those listed in Table I as SEQ ID NO: 16 through SEQ ID NO: 30.

The methods of the present invention are amenable to high throughput applications; however, the use of as few as one test compound in the method is encompassed by the term “screening”. Test compounds which bind to D₁ or D₅ dopamine receptors, activate D₁ or D₅ dopamine receptors, prolong or augment the agonist-induced activation of D₁ or D₅ dopamine receptors or of a D₁ or D₅ dopamine receptor signal transduction pathway, or increase expression of D₁ or D₅ dopamine receptors as determined by a method of the present invention, are referred to herein as “candidate compounds.” Such candidate compounds can be used to regulate skeletal muscle mass or function. However, more typically, this first level of in vitro screen provides a means by which to select a narrower range of compounds, i.e., the candidate compounds, which merit further investigation in additional levels of screening. The skilled artisan will recognize that a utility of the present invention is to identify, from a group of one or more test compounds, a subset of compounds which merit further investigation. One of skill in the art will also recognize that the assays of the present invention are useful in ranking the probable usefulness of a particular candidate compound relative to other candidate compounds. For instance, a candidate compound which activates D₁ or D₅ dopamine receptors at 1000 nM (but not at 10 nM) is of less interest than one which activates D₁ or D₅ dopamine receptors at 10 nM. Using such information the skilled artisan may select a subset of the candidate compounds, identified in the first level of screening, for further investigation. The skilled artisan will also recognize that, depending on how the group of test compounds is selected, and how the positives are selected, only a certain proportion of test compounds will be identified as candidate compounds, and that this proportion may be very small.

The assay systems described below may be formulated into kits comprising D₁ or D₅ dopamine receptors or cells expressing the D₁ or D₅ dopamine receptors which can be packaged in a variety of containers, e.g., vials, tubes microtitre well plates, bottles and the like. Other reagents can be included in separate containers and provided with the kit, e.g., positive control samples, negative control samples, buffers and cell culture media.

In one embodiment, the invention provides a method for screening one or more test compounds to identify candidate compounds that bind to either D₁ or D₅ dopamine receptors or both. Methods of determining binding of a compound to a receptor are well known in the art. Typically, the assays include the steps of incubating a source of the D₁ and D₅ dopamine receptors with a labeled compound, known to bind to the receptor, in the presence or absence of a test compound and determining the amount of bound labeled compound. The source of D₁ and D₅ dopamine receptors may either be cells expressing D₁ and D₅ dopamine receptors or some form of isolated D₁ and D₅ dopamine receptors, as described herein. The labeled compound can be dopamine or any dopamine analog (preferably a D₁ and D₅ dopamine receptor ligand including but not limited to SCH 23390) labeled such that it can be measured, preferably quantitatively (e.g., ¹²⁵I-labeled, 3H-labeled, 14C-labeled, europium labeled, fluorescein labeled). Such methods of labeling are well known in the art. Test compounds that bind to the dopamine receptor cause a reduction in the amount of labeled ligand bound to the receptor, thereby reducing the signal level compared to that from control samples (absence of test compound). Variations of this technique have been described in which receptor binding in the presence and absence of G-protein uncoupling agents can discriminate agonists from antagonists (e.g., binding in the absence and presence of a guanine nucleotide analog i.e., GpppNHp). See Keen, M., Radioligand Binding Methods for Membrane Preparations and Intact cells in Receptor Signal Transduction Protocols, R. A. J. Challis, (ed), Humana Press Inc., Totoway N.J. (1997).

Because it is desirable to discriminate between compounds which bind specifically to D₁ and D₅ dopamine receptors, as compared with, the assays described above should be conducted using a cell, or membrane from a cell, which expresses only D₁ and D₅ dopamine receptors or the assays can be conducted with a recombinant source of D₁ and D₅ dopamine receptors. Cells expressing both forms of dopamine receptor may be modified using homologous recombination to inactivate or otherwise disable one of the dopamine receptor genes. Alternatively, if the source of dopamine receptor contains more than one dopamine receptor type, the background signal produced by the receptor, which is not of interest, must be subtracted from the signal obtained in the assay. The background response can be determined by a number of methods, including elimination of the signal from the dopamine receptor, which is not of interest, by use of antisense, antibodies or selective antagonists. Known antagonists of dopamine receptors include SCH23390 (D₁ and D₅ dopamine receptors selective), and Spiperone (D_{2/3/4} dopamine receptors selective).

In another embodiment, the invention provides methods for screening test compounds to identify candidate compounds which activate D₁ and D₅ dopamine receptors. This could be used in conjunction with the binding assays described herein above. Typically, the assays are cell-based; however, cell-free assays are known which are able to differentiate agonist and antagonist binding as described above. Cell-based assays include the steps of contacting cells which express D₁ and D₅ dopamine receptors with a test compound or control and measuring activation of the dopamine receptor by measuring the expression or activity of components of the dopamine receptor signal transduction pathways.

As described in the background section above, dopamine receptors appear to couple through several different pathways including G_{αq} or G_{αi}, depending upon the cell type. It is thought that agonist activation of dopamine receptor allows the receptor to signal via any of these pathways, provided that the necessary pathway components are present in the particular cell type. Thus, to screen for dopamine receptor activation, an assay can use any of the signal transduction pathways as the readout even if the relevant cell type for treatment, in vivo, couples dopamine receptor to skeletal muscle atrophy via a different pathway. One of ordinary skill in the art would recognize that a screening assay would be effective for identifying useful dopamine receptor agonists independent of the pathway by which receptor activation was measured. Assays for measuring activation of these signaling pathways are known in the art.

For example, after contact with the test compound, lysates of the cells can be prepared and assayed for induction of cAMP. cAMP is induced in response to G_{αs} activation. Because G_{αs} is activated by receptors other than dopamine receptor and because a test compound may be exerting its effect through dopamine receptors or by another mechanism, two control comparisons are relevant for determining whether a text compound increases levels of cAMP via activation of a dopamine receptor. One control compares the cAMP level of cells contacted with a test compound and the cAMP level of cells contacted with a control compound (i.e., the vehicle in which the test compound is dissolved). If the test compound increases cAMP levels relative to the control compound this indicates that the test compound is increasing cAMP by some mechanism. The other control compares the cAMP levels of a dopamine receptor expressing cell line and a cell line that is essentially the same except that it does not express the dopamine receptor, where both of the cell lines have been treated with test compound. If the test compound elevates cAMP levels in the dopamine receptor expressing cell line relative to the cell line that does not express dopamine receptors, this is an indication that the test compound elevates cAMP via activation of the dopamine receptors.

In a specific embodiment of the invention, cAMP induction is measured with the use of DNA constructs containing the cAMP responsive element linked to any of a variety of reporter genes can be introduced into cells expressing dopamine receptors. Such reporter genes include, but are not limited to, chloramphenicol acetyltransferase (CAT), luciferase, glucuronide synthetase, growth hormone, fluorescent proteins (e.g., Green Fluorescent Protein), or alkaline phosphatase. Following exposure of the cells to the test compound, the level of reporter gene expression can be quantitated to determine the test compound's ability to increase cAMP levels and thus determine a test compounds ability to activate the dopamine receptor.

The cells useful in this assay are the same as for the dopamine receptor binding assay described above, except that cells utilized in the activation assays preferably express a functional receptor which gives a statistically significant response to dopamine or one or more dopamine analogs. In addition to using cells expressing full length dopamine receptors, cells can be engineered which express dopamine receptors containing the ligand binding domain of the receptor coupled to, or physically modified to contain, reporter elements or to interact with signaling proteins. For example, a wild-type dopamine receptor or dopamine receptor fragment can be fused to a G-protein resulting in activation of the fused G-protein upon agonist binding to the dopamine receptor portion of the fusion protein. (Siefert, R. et al., Trends Pharmacol. Sci. 20: 383-389 (1999)). The cells should also preferably possess a number of characteristics, depending on the readout, to maximize the inductive response by dopamine receptor or the dopamine receptor analog, for example, for detecting a strong induction of a CRE reporter gene; (a) a low natural level of cAMP; (b) G proteins capable of interacting with dopamine receptors; (c) a high level of adenylyl cyclase; (d) a high level of protein kinase A; (e) a low level of phosphodiesterases; and (f) a high level of cAMP response element binding protein would be advantageous. To increase the response to dopamine or a dopamine analog, host cells could be engineered to express a greater amount of favorable factors or a lesser amount of unfavorable factors. In addition, alternative pathways for induction of the CRE reporter could be eliminated to reduce basal levels.

In some instances, G protein-coupled receptor responses subside, or become desensitized, after prolonged exposure to an agonist. Another embodiment of the invention provides methods for identifying compounds that prolong or augment the agonist-induced activation of D₁ and D₅ dopamine receptors, or the D₁ and D₅ dopamine receptors signal transduction pathway, in response to a D₁ or D₅ dopamine receptors agonist. Such compounds may be used, for example, in conjunction with a D₁ or D₅ dopamine receptor agonist for the treatment of skeletal muscle atrophy. Typically the method uses a cell based assay comprising in any order or concurrently (i) contacting the cells with a test compound; (ii) treating cells expressing functional D₁ and D₅ dopamine receptors with D₁ and D₅ dopamine receptor agonists at a concentration of agonist and for a period of agonist-receptor exposure sufficient to allow desensitization of the receptor; followed by (iii) determining the level of activation of the D₁ and D₅ dopamine receptors. One of skill in the art will recognize that several mechanisms contribute to receptor desensitization including, but not limited to, receptor phosphorylation, receptor internalization or degradation and dopamine receptor signal transduction pathway down-modulation. One of skill in the art can determine the appropriate time (i.e., before, during or after agonist treatment) for contacting the cells with the test compounds depending upon which mechanism of desensitization is targeted. For example, contacting the cells with test compounds following agonist treatment, can detect test compounds which block receptor desensitization which occurs as a result of phosphorylation of the receptor.

In another embodiment, the invention provides a method of screening one or more test compound to identify candidate compounds which regulate transcription from the D₁ and D₅ dopamine receptor gene or regulate D₁ and D₅ dopamine receptor expression. Candidate compounds which regulate transcriptional activity of dopamine receptor genes may be identified using a reporter gene operably associated with D₁ and D₅ dopamine receptor regulatory region (reporter gene construct). Such methods are known in the art. In one such method, the reporter gene construct is contacted with a test compound in the presence of a source of cellular factors and the level of reporter gene expression is determined. A test compound which causes an increase in the level of expression, compared to a control sample, is indicative of a candidate compound which increases transcription of the D₁ and D₅ dopamine receptor genes. To provide the cellular factors required for in vitro or in vivo transcription, appropriate cells or cell extracts are prepared from any cell type that normally expresses D₁ and D₅ dopamine receptors.

Candidate compounds which regulate D₁ and D₅ dopamine receptor expression can also be identified in a method wherein a cell is contacted with a test compound and the expression of dopamine receptor is determined. The level of expression of D₁ and D₅ dopamine receptors in the presence of the test compound is compared with the level of expression in the absence of the test compound. Test compounds which increase the expression of D₁ and D₅ dopamine receptors are identified as candidate compounds for increasing muscle mass or muscle function. Such a method detects candidate compounds which increase the transcription or translation of the D₁ and D₅ dopamine receptors or which increase the stability of the mRNA or D₁ and D₅ dopamine receptor protein.

VII. Screening of Candidate Compounds Using Models of Skeletal Muscle Atrophy

Candidate compounds selected from one or more test compounds by an in vitro assay, as described above, can be further tested for their ability to regulate skeletal muscle mass or function in model systems of skeletal muscle atrophy and/or hypertrophy. Such models of skeletal muscle atrophy or hypertrophy include both in vitro cell culture models and in vivo animal models of skeletal muscle atrophy. Such additional levels of screening are useful to further narrow the range of candidate compounds that merit additional investigation, e.g., clinical trials.

### Cell Culture Models of Muscle Atrophy

In vitro models of skeletal muscle atrophy are known in the art. Such models are described, for example, in Vandenburgh, H. H., In Vitro 24:609-619 (1988), Vandenburgh, H. H. et al., J of Biomechanics, 24 Suppl 1:91-99 (1991), Vandenburgh, H. H et al., In Vitro Cell. Dev. Biol., 24(3):166-174 (1988), Chromiak, J. A., et al., In Vitro Cell. Dev. Biol. Anim., 34(9):694-703 (1998), Shansky, J., et al., In Vitro Cell. Dev. Biol. Anim., 33(9):659-661 (1997), Perrone, C. E. et al., J. Biol. Chem. 270(5):2099-2106 (1995), Chromiac, J. A. and Vandenburgh, H. H., J. Cell. Physiol. 159(3):407-414 (1994), and Vandenburgh, H. H. and Karlisch, P., In Vitro Cell. Dev. Biol. 25(7):607-616 (1989). Such models are useful, but not required; following the in vitro screening described above in order to further narrow the range of candidate compounds that merit testing in an animal model. Cell culture models are treated with candidate compounds and the response of the model to the treatment is measured by assessing changes in muscle markers such as: muscle protein synthesis or degradation, changes in skeletal muscle mass or contractile function. Those compounds which induce significant changes in the muscle markers are typically screened further in an animal model of skeletal muscle atrophy.

### Animal Models of Skeletal Muscle Atrophy

The candidate compounds are administered to non-human animals and the response of the animals is monitored, for example, by assessing changes in markers of atrophy or hypertrophy such as: skeletal muscle mass, skeletal muscle function, muscle or myofiber cross-sectional area, contractile protein content, non-contractile protein content or a biochemical or genetic marker that correlates with skeletal muscle mass or function changes. Candidate compounds which induce skeletal muscle hypertrophy or prevent any aspect of skeletal muscle atrophy should be considered as prospective therapeutic candidates for treatment of human skeletal muscle atrophy, and are referred to herein as candidate therapeutic compounds. In addition to assessing the ability of a candidate compound to regulate skeletal muscle atrophy, undesirable side effects such as toxicity may also be detected in such a screen. The absence of unacceptably high levels of side effects may be used as a further criterion for the selection of candidate therapeutic compounds.

A variety of animal models for skeletal muscle atrophy are known in the art, such as those described in the following references: Herbison, G. J., et al. Arch. Phys. Med. Rehabil. 60:401-404 (1979), Appell, H-J. Sports Medicine 10:42-58 (1990), Hasselgren, P-O. and Fischer, J. E. World J. Surg. 22:203-208 (1998), Agbenyega, E. T. and Wareham, A. C. Comp. Biochem. Physiol. 102A: 141-145 (1992), Thomason, D. B. and Booth, F. W. J. Appl. Physiol. 68:1-12 (1990), Fitts, R. H., et al. J. Appl. Physiol. 60:1946-1953 (1986), Bramanti, P., et al. Int. J. Anat. Embryol. 103:45-64 (1998), Cartee, G. D. J. Gerontol. A Biol. Sci. Med. Sci. 50:137-141 (1995), Cork, L. C., et al. Prog. Clin. Biol. Res. 229:241-269 (1987), Booth, F. W. and Gollnick, P. D. Med. Sci. Sports Exerc. 15:415-420 (1983), Bloomfield, S. A. Med. Sci. Sports Exerc. 29:197-206 (1997). Preferred animals for these models are mice and rats. These models include, for example, models of disuse-induced atrophy such as casting or otherwise immobilizing limbs, hind limb suspension, complete animal immobilization, and reduced gravity situations. Models of nerve damage induced atrophy include, for example, nerve crush, removal of sections of nerves which innervate specific muscles, toxin application to nerves and infection of nerves with viral, bacterial or eukaryotic infectious agents. Models of glucocorticoid-induced atrophy include application of atrophy-inducing doses of exogenous glucocorticoid to animals, and stimulation of endogenous corticosteroid production, for example, by application of hormones that activate the hypothalamus-pituitary-adrenal (HPA) axis. Models of sepsis-induced atrophy include, for example, inoculation with sepsis-inducing organisms such as bacteria, treatment of the animal with immune-activating compounds such as bacterial cell wall extract or endotoxin, and puncture of intestinal walls. Models of cachexia-induced atrophy include, for example, inoculation of an animal with tumorigenic cells with cachexia forming potential, infection of an animal with infectious agents (such as viruses which cause AIDS) which result in cachexia and treatment of an animal with hormones or cytokines such as CNTF, TNF, IL-6, IL-1, etc. which induce cachexia. Models of heart failure-induced atrophy include the manipulation of an animal so that heart failure occurs with concomitant skeletal muscle atrophy. Neurodegenerative disease-induced atrophy models include autoimmune animal models such as those resulting from immunization of an animal with neuronal components. Muscular dystrophy-induced models of atrophy include natural or man-made genetically-induced models of muscular dystrophy such as the mutation of the dystrophin gene which occurs in the Mdx mouse.

Animal models of skeletal muscle hypertrophy include, for example, models of increased limb muscle use due to inactivation of the opposing limb, reweighting following a disuse atrophy inducing event, reutilization of a muscle which atrophied because of transient nerve damage, increased use of selective muscles due to inactivation of a synergistic muscle (e.g., compensatory hypertrophy), increased muscle utilization due to increased load placed on the muscle and hypertrophy resulting from removal of the glucocorticoid after glucocorticoid-induced atrophy. Preferred animal atrophy models include the sciatic nerve denervation atrophy model, glucocorticoid-induced atrophy model, and the leg casting disuse atrophy model that are described in further detail below.

The sciatic nerve denervation atrophy model involves anesthetizing the animal followed by the surgical removal of a short segment of either the right or left sciatic nerve, e.g., in mice the sciatic nerve is isolated approximately at the midpoint along the femur and a 3-5 mm segment is removed. This denervates the lower hind limb musculature resulting in atrophy of these muscles. Typically, innervation to the biceps femoris is left intact to provide satisfactory motion of the knee for virtually normal ambulation. Typically, in untreated animals, muscle mass of the denervated muscles is reduced 30-50% ten days following denervation. Following denervation, test compounds are administered e.g., by injection or by continuous infusion, e.g., via implantation of an osmotic minipump (e.g., Alzet, Palo Alto, Calif.), to determine their effect on denervation induced skeletal muscle atrophy. At various times following denervation, the animals are euthanized and lower leg muscles are dissected rapidly from both the denervated and nondenervated legs, the muscles, cleaned of tendons and connective tissue, are weighed. The extent of atrophy in the affected muscles is analyzed, for example, by measuring muscle mass, muscle cross-sectional area, myofiber cross-sectional area or contractile protein content.

The glucocorticoid-induced atrophy model involves the administration of a glucocorticoid to the test animal, e.g., 1.2 mg/kg/day of dexamethasone in the drinking water. Typically, in untreated animals, skeletal muscle mass is reduced 30-50% following ten days of dexamethasone administration. Concomitantly with, or following glucocorticoid administration, test compounds are administered e.g., by injection or by continuous infusion to determine their effect on glucocorticoid-induced skeletal muscle atrophy. At various times following glucocorticoid administration, the extent of atrophy in the affected muscles is analyzed as described above for the denervation model.

The leg casting disuse atrophy model involves casting one hind leg of an animal from the knee down through the foot. Typically, muscle mass is reduced 20-40% after ten days of casting. Following casting, test compounds are administered by injection or by continuous infusion via implantation of an osmotic minipump (e.g., Alzet, Palo Alto, Calif.) to determine their effect on leg casting induced skeletal muscle atrophy. At various times following leg casting, the extent of atrophy in the affected muscles is analyzed as described above for the denervation model.

One of skill in the art would recognize that in screening for compounds for human use, because there are differences between the human D₁ and D₅ dopamine receptors and the D₁ and D₅ dopamine receptors from other animal species, there may be some false positive or negative results which arise when the screen is carried out using non-human D₁ and D₅ dopamine receptors. Thus, it is preferable to do the initial in vitro screen using human D₁ and D₅ dopamine receptors. In certain circumstances, identified candidate compounds may be active toward only the human receptor and not toward a non-human receptor. In such circumstances, it may still be desirable to determine whether these candidate compounds are able to regulate skeletal muscle mass or function in a second level of screening. Because these candidates do not activate non-human D₁ and D₅ dopamine receptors, a standard in vivo screen with non-human animal is not advised. In such circumstances the second level of screening for these candidates may be performed in transgenic animals that express human dopamine receptors.

Animals of any species, especially mammals, including, but not limited to, mice, rats, rabbits, guinea pigs, pigs, goats, dogs and non-human primates may be used to generate dopamine receptor transgenic animals. Mice and rats are preferred, mice are most preferred. A variety of techniques are known in the art and may be used to introduce the human dopamine receptor transgenes into animals to produce the founder lines of transgenic animals. Such techniques include, but are not limited to, pronuclear microinjection, retrovirus-mediated gene transfer into germ lines, gene targeting in embryonic stem cells, electroporation of embryos and sperm-mediated gene transfer.

VIII. Gene Therapy Methods for the Treatment of Skeletal Muscle Atrophy

The overall activity of D₁ and D₅ dopamine receptors can be increased by overexpressing a gene for D₁ and D₅ dopamine receptors (to increase expression of D₁ and D₅ dopamine receptors) or a constitutively active D₁ and D₅ dopamine receptors in the appropriate tissue. Dopamine receptor levels can be increased, in vivo, by likewise overexpressing a dopamine receptor gene. Overexpression of these genes will increase the total cellular D₁ and D₅ dopamine receptor activity, thus, regulating skeletal muscle atrophy. The gene or genes of interest are inserted into a vector suitable for expression in the subject. These vectors include, but are not limited to, adenovirus, adenovirus associated virus, retrovirus and herpes virus vectors in addition to other particles that introduced DNA into cells (e.g., liposome, gold particles, etc.) or by direct injection of the DNA expression vector, containing the gene of interest, into human tissue (e.g., muscle).

IX. Pharmaceutical Formulations and Methods for Use

Candidate compounds or candidate therapeutic compounds identified by screening methods described herein can be administered to individuals to treat skeletal muscle atrophy, or to induce skeletal muscle hypertrophy. To this end, the present invention encompasses methods and compositions for modulating skeletal muscle atrophy, including, but not limited to, skeletal muscle atrophy induced by disuse due to surgery, bed rest, broken bones; denervation/nerve damage due to spinal cord injury; autoimmune disease; infectious disease; glucocorticoid use for unrelated conditions; sepsis due to infection or other causes; nutrient limitation due to illness or starvation; cancer cachexia; chronic inflammation; AIDS cachexia; COPD; congestive heart failure; sarcopenia and genetic disorders; e.g., muscular dystrophies, neurodegenerative diseases. Agonists of D₁ and D₅ dopamine receptors can be used to inhibit skeletal muscle atrophy. It is not necessary that effective compounds demonstrate absolute specificity for dopamine receptor. It is contemplated that specific antagonist of other affected receptors can be co-administered with an effective, but nonspecific, agonist. Alternately, this lack of specificity may be addressed by modulation of dose alone, or the dosing regimen.

The candidate compounds or candidate therapeutic compounds identified by the screening methods of the present invention may be administered in conjunction with compounds which prolong or augment the activation of a D₁ and D₅ dopamine receptors or of a D₁ and D₅ dopamine receptors signal transduction pathway. These may be known compounds, for example, theophylline, or these compounds may be identified by the screening methods of this invention to prolong or augment the activation of a D₁ and D₅ dopamine receptor or of a D₁ and D₅ dopamine receptor signal transduction pathway.

### Dose Determinations

Safety and therapeutic efficacy of compounds which agonize dopamine receptor can be determined by standard procedures using either in vitro or in vivo technologies. Compounds which exhibit large therapeutic indices are preferred, although compounds with lower therapeutic indices are useful if the level of side effects is acceptable. The data obtained from the in vitro and in vivo toxicological and pharmacological techniques can be used to formulate the human range of doses which may be useful. The preferred dose lies in the range in which the circulating concentration of the compound is therapeutically maximal with acceptable safety. The circulating concentration of the compound may vary depending on the dose form, time after dosing, route of administration, etc. Doses outside this range are also useful provided the side effects are acceptable. Such matters as age and weight of the patient, and the like, can be used to determine such matters in the conventional manner. Pharmacogenetic approaches may be useful in optimizing compound selection, doses and dosing regimen in clinical populations.

### Formulation and Use

Pharmaceutical compositions for use in the modulation of skeletal muscle atrophy in accordance with the present invention may be formulated using conventional methodologies using pharmaceutically acceptable carriers and excipients. The compositions of this invention are preferably provided in unit dosage form. As used herein, a “unit dosage form” is a composition of this invention containing an amount of a D₁ and D₅ dopamine receptor agonist that is suitable for administration to an animal, preferably a mammal, more preferably a human subject, in a single dose, according to good medical practice. Pharmaceutical compositions may be formulated for delivery by, for example, intranasal, transdermal, inhalation, parenteral, cutaneous, oral or rectal administration. For oral administration, the pharmaceutical composition may take the form of tablets or capsules containing the pharmacologically active compound and additives including, but not limited to, binding agents, fillers, lubricants, disintegrants, or wetting agents. The tablets may be coated. Liquid preparations for oral administration include, but are not limited to, syrups, suspensions or dry products which are reconstituted with liquid vehicle before use, containing the pharmacologically active compound and additives including, but not limited to, suspending agents, emulsifying agents, non-aqueous vehicles, preservatives, buffer salts, flavoring, coloring, sweetening agents, etc. Pharmaceutical compositions for oral administration may be formulated for controlled release of the pharmacologically active compounds either in the mouth, stomach or intestinal tract.

For inhalation administration, the compounds for use according to the present invention may be delivered by, but not limited to, the following forms: liquid, powder, gel or in the form of an aerosol spray utilizing either pressurized or non-pressurized propellants in either premeasured or non-premeasured doses. The pharmacologically active compound may be formulated with appropriate fillers, vehicles, preservatives, buffers, etc. For parenteral administration, the pharmacologically active compound may be formulated with acceptable physiological carriers, preservatives, etc. and be prepared as suspensions, solutions, emulsion, powders ready for constitution, etc. for either bolus injection or infusion. Doses of these compounds may be administered by a variety of technologies including hypodermic needles, high-pressure devices, etc. For rectal administration, the pharmacologically active compound may be formulated with acceptable physiological carriers, preservatives, etc. for delivery as suppositories, enemas, etc. For cutaneous administration, the pharmacologically active compound may be formulated with acceptable physiological carriers including lotions, emollients, etc. or incorporated into a patch type device. For long-term administration, the pharmacologically active compound and appropriate additives such as, but limited to, polymers, hydrophobic materials, resins, etc. may be formulated as a depot preparation for either injection or implantation at multiple sites including but not limited to intramuscular and subcutaneous locations. In addition, the pharmacologically active compound may be administered by a dispensing device.

Monitoring of Effects During Clinical Trials

Monitoring the influence of compounds (e.g., drugs) on the expression or activity of D₁ and D₅ dopamine receptors can be employed not only in basic drug screening, but also in clinical trials. For example, the effectiveness of a compound determined by a screening assay to increase D₁ and D₅ dopamine receptor activity or D₁ and D₅ dopamine receptor expression can be assessed in clinical trials of patients with, or at risk for, skeletal muscle atrophy. At various times following administration of the test compound or placebo, the effect of the compound on the patient can be determined, for example, by observing the change in skeletal muscle mass, skeletal muscle function, biochemical markers of muscle breakdown or quality of life measures. Methods of measuring skeletal muscle mass in human subjects are known in the art and include, for example: measuring the girth of a limb; measuring muscle thickness with for instance, computer tomography, MRI or supersonics; or muscle biopsy to examine morphological and biochemical parameters (e.g., cross-section fiber area, fiber diameter or enzyme activities). Furthermore, because skeletal muscle mass is correlated with skeletal muscle function, muscle function can be used as a surrogate marker of mass and muscle mass changes can be assessed using functional measurements, e.g., strength, the force of a group of synergist muscles, or contraction characteristics found in electromyographic recordings. In addition, muscle protein loss as a result of muscle atrophy can be measured by quantitating levels of amino acids or amino acids derivatives, i.e., 3-methyl histidine, in the urine or blood of a subject. For a review of such methods see Appell, Sports Med. 10:42-58 (1990). Quality of life measures include, but are not limited to, the ease of getting out of a chair, number of steps taken before tiring or ability to climb stairs.

### EXAMPLES

### Example 1

### Construction of Vectors for Human D₁ and D₅ Dopamine Receptors Expression

The human D₁ and D₅ dopamine receptors (hD₁ and hD₅ dopamine receptors) DNA sequences, Accession No. X58987 and X58454, are retrieved and two oligonucleotides including one containing the 5′ end of the gene beginning at the initiation codon (5′ oligonucleotide) and one containing the 3′ end of the gene containing the stop codon (3′ oligonucleotide) are synthesized. These oligonucleotides are designed to contain restriction endonuclease sites which are not present in the D₁ or D₅ dopamine receptor gene with one unique site in the 5′ oligonucleotide and a different unique restriction endonuclease site in the 3′ oligonucleotide. In addition, the 3′ oligonucleotide contains a polyadenylation addition signal sequence. Double stranded cDNA from human skeletal muscle is purchased from the Universal QUICK-Clone cDNA collection (Clonetech Inc., Palo Alto, Calif., USA). Using the above 5′ and 3′ oligonucleotides, the D₁ and D₅ dopamine receptors cDNA is amplified by PCR of the human skeletal muscle cDNA using the AdvanTaq PCR kit (Clonetech Inc., Palo Alto, Calif., USA). The D₁ and D₅ dopamine receptor gene PCR product is purified from PCR artifacts by agarose gel electrophoresis and the D₁ and D₅ dopamine receptor gene DNA fragment is purified from the agarose gel using a purification product such as NucleoTrap (Clonetech Inc., Palo Alto, Calif., USA).

Cloning of the D₁ and D₅ dopamine receptor PCR products into the pIRESneo vector (Clonetech Inc., Palo Alto, Calif., USA) is accomplished by first cutting the D₁ and D₅ dopamine receptor PCR product and the pIRESneo vector with the appropriate restriction endonucleases so that the 5′ and 3′ restriction endonuclease sites are ready for ligation. The pIRESneo vector DNA is ligated to the D₁ and D₅ dopamine receptor PCR products DNA using DNA ligase, from the AdvantAge™ PCR Cloning Kit (Clonetech Inc., Palo Alto, Calif., USA), according to the manufacturer's recommendations. The ligated vector and insert construct (pIRESneo/D₁ and D₅ dopamine receptors) is then used to transform TOP10F′ competent E. coli cells (Clonetech Inc., Palo Alto, Calif., USA). Transformed cells are plated on LB/X-gal/IPTG plus ampicillin containing agar. White colonies (positive clones) are selected and individually cultured in LB medium. Plasmid DNA is isolated using NucleoBond DNA Purification System (Clonetech Inc., Palo Alto, Calif., USA). The insert from at least one clone is sequenced to ensure that the D₁ and D₅ dopamine receptor sequence is correct. HEK293 cells containing a stably integrated Mercury CRE-LUC plasmid (Clonetech Inc., Palo Alto, Calif., USA) are transfected with purified pIRESneo/D₁ and D₅ dopamine receptors DNA, having the correct sequence insert, utilizing the CalPhos™ Mammalian Transfection Kit (Clonetech Inc., Palo Alto, Calif., USA. Cells stably transfected with pIRESneo/D₁ and D₅ dopamine receptors DNA are selected by culturing the cells in G418. The stably transfected cells (HEK293/CRE-LUC/pIRESneo/D₁ and D₅ dopamine receptors cells) are propagated in DMEM (Life Technologies, Rockville, Md.) containing 10% fetal bovine serum (Clonetech Inc., Palo Alto, Calif., USA), penicillin/streptomycin solution (Life Technologies, Rockville, Md.), L-glutamine (Life Technologies, Rockville, Md.), and non-essential amino acid (Life Technologies, Rockville, Md.) at 37° C. in a 5% carbon dioxide/95% air atmosphere. The clones are characterized for both dopamine receptor binding and CRE-LUC activation following exposure to dopamine receptor as described in Example 2 and Example 3. Cells expressing the D₁ and D₅ dopamine receptor at an appropriate level and which are appropriately coupled to the CRE-LUC reporter system are then utilized for further analysis.

### Example 2

### Receptor Binding Assays

Receptor binding analysis of compounds is performed in whole cells by plating the HEK293/CRE-LUC/pIRESneo/D₁ or D₅ dopamine receptors cells from Example 1 in a 96 well polylysine coated plate. Cells are seeded in DMEM medium containing 10% fetal bovine serum, penicillin/streptomycin solution, L-glutamine, and non-essential amino acid at 37° C. in a 5% carbon dioxide/95% air atmosphere and incubated overnight. The culture medium is removed and the appropriate amount of 3H-SCH23390 in MEM (Life Technologies, Rockville, Md.)+10% Seablock (Clonetech Inc., Palo Alto, Calif., USA) is added. The cells are incubated with the 3H-SCH23390 for 90 minutes at room temperature then washed 4 times with phosphate buffered saline lacking magnesium and calcium (Life Technologies, Rockville, Md.). Following the final wash, cytoscint es scintillation fluid is added (ICN Biomedical, Inc., Costa Mesa, Calif.) and the plate is read on a TopCount NXT Microplate Scintillation Counter (Packard Instrument Company, Meriden, Conn.). For saturation binding analysis, log doses of ranging from 10(−12) to 10(−3) M are added to the cells and binding analyzed both in the absence and the presence of a saturating concentration of SCH23390 for evaluation of non-specific binding. For competitive binding, a concentration of SCH23390 is added which is half maximal, in terms of binding, in addition to varying concentrations of the compound of interest.

### Example 3

### Receptor Activation Assay

Receptor activation analysis is performed by seeding the HEK293/CRE-LUC/pIRESneo/D₁ or D₅ dopamine receptors cells of Example 1 into Packard View Plate-96 (Packard Inc., CA). Cells are seeded in DMEM medium containing 10% fetal bovine serum, penicillin/streptomycin solution, L-glutamine, and non-essential amino acid at 37° C. in a 5% carbon dioxide/95% air atmosphere and incubated overnight. The medium is then removed and replaced with DMEM (Life Technologies, Rockville, Md.) containing 0.01% bovine albumin fraction V (SIGMA, St. Louis, Mo.) containing the compound of interest. The cells are then incubated for four hours at 37° C. in a 5% carbon dioxide/95% air atmosphere after which the medium is removed and the cells are washed twice with Hanks Balanced Salt Solution (Life Technologies, Rockville, Md.). Lysis Reagent (Promega Inc., Madison, Wis.) is then added to the washed cells and the cells are incubated for 20 minutes at 37° C. in a 5% carbon dioxide/95% air atmosphere. The cells are then placed at −80° C. for 20 minutes followed by a 20 minute incubation at 37° C. in a 5% carbon dioxide/95% air atmosphere. After this incubation, Luciferase Assay Buffer and Luciferase Assay Substrate (Promega Inc., Madison, Wis.) are added to the cell lysates and luciferase activity quantitated using a luminometer. Relative activity of a compound is evaluated by comparing the increase following exposure to compound to the level of luciferase in HEK cells which contain the CRE-LUC construct without the D₁ and D₅ dopamine receptors following exposure to compound. Specificity of response is also checked by evaluating luciferase response of D₁ and D₅ dopamine receptors/CRE-LUC HEK cells to compound in the presence and absence of a 10-fold excess of D₁ and D₅ dopamine receptors antagonist.

### Example 4

### Screen to Identify Candidate Compounds that Prolong or Augment the Activation of D₁ or D₅ Dopamine Receptors and/or a D₁ and D₅ Dopamine Receptor Signal Transduction Pathway

Identification of compounds that prolong or augment the agonist-induced activation of the D₁ or D₅ dopamine receptors or of a D₁ and D₅ dopamine receptors signal transduction pathway, involves a variation of the Receptor Activation Assay described in Example 3. Specifically, this assay is performed by seeding the HEK293/CRE-LUC/pIRESneo/D₁ and D₅ dopamine receptor cells into Packard View Plate-96 (Packard Inc., CA). Cells are seeded in DMEM medium containing 10% fetal bovine serum, penicillin/streptomycin solution, L-glutamine, non-essential amino acid, and saturating amounts of dopamine receptor at 37° C. in a 5% carbon dioxide/95% air atmosphere and incubated for 48 hours. The medium is then removed and replaced with DMEM (Life Technologies, Rockville, Md.) containing 0.01% bovine albumin fraction V (SIGMA, St. Louis, Mo.) and SKF81297 in addition to the compound of interest. The cells are then incubated for four hours at 37° C. in a 5% carbon dioxide/95% air atmosphere after which the medium is removed and the cells are washed twice with Hanks Balanced Salt Solution (Life Technologies, Rockville, Md.). Lysis Reagent (Promega Inc., Madison, Wis.) is then added to the washed cells and the cells are incubated for 20 minutes at 37° C. in a 5% carbon dioxide/95% air atmosphere. The cells are then placed at −80° C. for 20 minutes followed by a 20 minute incubation at 37° C. in a 5% carbon dioxide/95% air atmosphere. After this incubation, Luciferase Assay Buffer and Luciferase Assay Substrate (Promega Inc., Madison, Wis.) are added to the cell lysates and luciferase activity is quantitated using a luminometer. Test compounds which stimulate fluorescence significantly above the levels of control untreated cells, after correction for variations in cell density, are considered candidate compounds for regulating skeletal muscle mass or function.

The compounds of most interest are those that induce relatively higher levels of fluorescence.

### Example 5

### Screens to Identify Candidate Compounds that Increase D₁ or D₅ Dopamine Receptor Expression

The sequence containing the promoter region of the D₁ or D₅ dopamine receptor genes, beginning far enough upstream of the transcriptional initiation site to contain all the regulatory elements necessary for physiological expression of the D₁ or D₅ dopamine receptor genes in the appropriate tissue is retrieved from the human genome database. Two oligonucleotides, one containing the 5′ end of the promoter region (5′ oligonucleotide) and one containing the 3′ end of the promoter region including the transcriptional start site (3′ oligonucleotide) are synthesized. These oligonucleotides also contain restriction endonuclease sites which are not present in the D₁ or D₅ dopamine receptor genes regulatory region with one unique site in the 5′ oligonucleotide and a different unique restriction endonuclease site in the 3′ oligonucleotide. The 5′ and 3′ oligonucleotides are used for PCR amplification of the D₁ or D₅ dopamine receptor genes regulatory region from human DNA (Clonetech Inc., Palo Alto, Calif., USA) using the PCR kit, Advantage®Genomic PCR kit (Clonetech Inc., Palo Alto, Calif., USA). The D₁ and D₅ dopamine receptor genes regulatory region PCR products are purified from PCR artifacts by agarose gel electrophoresis and the D₁ and D₅ dopamine receptor genes regulatory region DNA fragment is purified from the agarose gel using a purification product such as NucleoTrap (Clonetech Inc., Palo Alto, Calif., USA). Cloning of the D₁ and D₅ dopamine receptor genes regulatory region PCR products into the pECFP-1 vector (Clonetech Inc., Palo Alto, Calif., USA) is accomplished by first cutting the D₁ and D₅ dopamine receptor genes regulatory region PCR products and the pECFP-1 vector with the appropriate restriction endonucleases so that the 5′ and 3′ restriction endonuclease sites are ready for ligation. Ligation of the pECFP-1 vector DNA to the D₁ and D₅ dopamine receptor genes regulatory region PCR products DNA are accomplished using DNA ligase from the AdvantAge™PCR Cloning Kit (Clonetech Inc., Palo Alto, Calif., USA) according to the manufacturer's recommendations. The ligated vector and insert construct is then used to transform TOP10F′ competent E. coli cells (Clonetech Inc., Palo Alto, Calif., USA). The cells are plated on LB plus kanamycin containing agar and kanamycin resistant colonies are selected for further analysis. Kanamycin resistant clones are cultured in LB containing kanamycin medium and plasmid DNA is isolated using NucleoBond DNA Purification System (Clonetech Inc., Palo Alto, Calif., USA) and the construct containing the D₁ and D₅ dopamine receptor genes regulatory region is analyzed by DNA sequencing to ensure construct correctness and integrity. Purified construct plasmid DNA containing the D₁ and D₅ dopamine receptor genes regulatory region is then transfected into the HEK293 cells utilizing calcium phosphate-mediated transfection utilizing the CalPhos™ Mammalian Transfection Kit (Clonetech Inc., Palo Alto, Calif., USA). Transfected cell clones are selected using G418, isolated and propagated in DMEM (Life Technologies, Rockville, Md.) containing 10% fetal bovine serum (Clonetech Inc., Palo Alto, Calif., USA), penicillin/streptomycin solution (Life Technologies, Rockville, Md.), L-glutamine (Life Technologies, Rockville, Md.), non-essential amino acid (Life Technologies, Rockville, Md.) and G418 (Life Technologies, Rockville, Md.) at 37° C. in a 5% carbon dioxide/95% air atmosphere. G418 resistant clones are characterized by Southern blotting to ensure that they contain the D₁ and D₅ dopamine receptor genes promoter sequence; in addition activation of the D₁ and D₅ dopamine receptor genes regulatory region is analyzed using an appropriate stimulating agent. Cells expressing the D₁ and D₅ dopamine receptor genes regulatory region-ECFP at an appropriate level are then used in assays designed to evaluate compounds which can modulate the activity of the D₁ and D₅ dopamine receptor genes regulatory region as follows. The regulatory region activation analysis is performed by seeding the D₁ and D₅ dopamine receptor genes regulatory region-ECFP containing HEK293 cells at an appropriate density into black with clear bottom 96 well microtiter plates and allowed to grow overnight. The following day, the medium is removed and the test compound added in fresh growth medium. The cells are incubated for 16 hours at 37° C. in a 5% carbon dioxide/95% air atmosphere followed by measurement of fluorescence (excitation at 433 (453) nm by detecting emission at 475(501) nm using a fluorometer (biolumin™ 960, Molecular Dynamics/Amersham Pharmacia Biotech, Piscataway, N.J.). Test compounds which stimulate fluorescence significantly above the levels of control untreated cells, after correction for variations in cell density, are considered candidate compounds for regulating skeletal muscle mass or function. The compounds of most interest are those which induce relatively higher levels of fluorescence.

### Example 6

### Method of Making Human Antibodies which Activate the D₁ and D₅ Dopamine Receptors

Fully human monoclonal antibodies which activate the D₁ and D₅ dopamine receptors are produced by first generating recombinant D₁ and D₅ dopamine receptor proteins as follows. The procedure from Example 1 is followed to obtain the D₁ and D₅ dopamine receptors PCR product. This D₁ and D₅ dopamine receptors PCR product is then cloned into the pHAT20 vector (Clonetech Inc., Palo Alto, Calif., USA) by first cutting the D₁ and D₅ dopamine receptor gene PCR product and the pHAT20 vector with the appropriate restriction endonucleases so that the 5′ and 3′ restriction endonuclease sites are ready for ligation. Ligation of the pHAT20 vector DNA to the D₁ and D₅ dopamine receptor genes PCR product DNA is accomplished using DNA ligase from the AdvantAge™PCR Cloning Kit (Clonetech Inc., Palo Alto, Calif., USA) according to the manufacturer's recommendations. The ligated vector/insert construct is then used to transform TOP10F′ competent E. coli cells (Clonetech Inc., Palo Alto, Calif., USA). Transformed cells are plated on LB plus ampicillin containing agar and ampicillin resistant colonies are selected for further analysis. Positive clones are cultured in LB medium containing ampicillin and plasmid DNA is isolated using NucleoBond DNA Purification System (Clonetech Inc., Palo Alto, Calif., USA) and the construct containing the D₁ and D₅ dopamine receptor genes is analyzed by DNA sequencing the ensure construct correctness and integrity. The D₁ and D₅ dopamine receptors-pHAT20 vector DNA is then used for additional PCR cloning by utilizing a 5′ oligonucleotide containing the beginning of the HAT sequence and a unique restriction endonuclease site not present in the D₁ and D₅ dopamine receptors-pHAT20 construct and the 3′ D₁ and D₅ dopamine receptors oligonucleotide utilized previously. The oligonucleotide primers are used to PCR amplify the HAT-D₁ and D₅ dopamine receptors fusion gene from the D₁ and D₅ dopamine receptors-pHAT20 construct and the PCR product is purified as described above. The HAT-D₁ and D₅ dopamine receptors fusion gene PCR product is then utilized for cloning into the pBacPAK8 vector using the BacPAK Baculovirus Expression System from Clonetech (Clonetech Inc., Palo Alto, Calif., USA). The ligation of the HAT-D₁ and D₅ dopamine receptors fusion gene into the pBacPAK8 vector is essentially as described above. The D₁ and D₅ dopamine receptors/HAT-pBacPAK8 construct is then transfected into TOP10′F competent E. coli cells, ampicillin resistant cells are selected and plasmid DNA is isolated and checked for construct integrity as described above. This construct is then cotransfected with linearized BacPAK6 DNA into Sf21 insect host cells utilizing the CalPhos™ Mammalian Transfection Kit (Clonetech Inc., Palo Alto, Calif., USA). The insect cells are then incubated for 2-3 days followed by harvest of virus from individual clear plaques. The virus is then amplified in Sf21 cells, the harvested virus titered, and the titered virus used for large scale infection of Sf21 cells utilizing BacPAK Insect Cell Media—all according to the manufacturers recommendations (Clonetech Inc., Palo Alto, Calif., USA). Recombinant HAT-D₁ and D₅ dopamine receptor fusion proteins are then purified using the TALON® CellThru Purification Kit from Clonetech (Clonetech Inc., Palo Alto, Calif., USA) using conditions recommended by the manufacturer. Briefly, infected Sf21 cells are harvested 48 hours after infection and sonicated in extraction/loading buffer. The cell lysate is then put through a TALON® CellThru column. The column is washed twice with extraction/loading buffer and the bound HAT-D₁ and D₅ dopamine receptor proteins are eluted with elution buffer. The eluted protein is analyzed by SDS-PAGE for integrity and protein concentration is quantitated using the Bio-Rad SDS-PAGE system and protein quantitation systems according to the manufacturer's recommendations (Bio-Rad Laboratories, Hercules, Calif.). Purified HAT-D₁ and D₅ dopamine receptor fusion proteins are then used for immunizing XenoMouse animals (Abgenix Inc., Fremont, Calif.) for human monoclonal antibody production as follows. 10 μg of purified recombinant HAT-D₁ and D₅ dopamine receptor fusion proteins in combination with 25 μg of adjuvant monophosphoryl lipid A (Sigma, St. Louis, Mo.) is used to vaccinate 10 XenoMouse animals multiple times over an eight week period. Serum is obtained from vaccinated animals and utilized in an antigen capture ELISA utilizing purified HAT-D₁ and D₅ dopamine receptor fusion proteins to detect antibodies to the HAT-D₁ and D₅ dopamine receptor proteins by coating polystyrene ELISA plates (Corning Glass Works, Corning, N.Y.) with HAT-D₁ and D₅ dopamine receptor fusion proteins, blocked with PBS-1% BSA, washed and incubated at 37° C. for 1 hour with a 1:50 dilution of the serum samples. After washing 5 times with PBS, the plates are incubated at 37° C. for 1 hour with alkaline phosphatase-conjugated goat antibodies to human immunoglobulin G. The plates are then washed 5× with PBS and antibodies detected with p-nitrophenyl phosphate substrate (Sigma, St. Louis, Mo.) in buffer. Optical densities at 405 nm were measured using a plate reader and signal quantitated. Mice with demonstrated high antibody production are used for hybridoma formation. Hybridomas are generated by fusion of splenic cells from the XenoMouse animals with nonsecreting myeloma cell line NSA-bcl 2 using a 4:1 ratio of spleen cells to NSA-bcl2 cells in the presence of 30% polyethylene glycol PEG 1450. Fused cells are individually cloned by limiting dilution into 96 well plates and cultured in RPMI-1640 medium containing 10% fetal bovine serum, nonessential amino acids, sodium pyruvate, L-glutamine, 100 u/ml penicillin-streptomycin and hypoxanthine-aminopterin-thymidine (all from Life Technologies, Rockville, Md.). Supernatants from the hypoxanthine-aminopterin-thymidine selected hybridomas were screened for human antibody production by ELISA as described previously. Hybridomas which produce human antibodies to the HAT-D₁ and D₅ dopamine receptors fusion protein are selected for large scale antibody production. Monoclonal antibodies are purified by Protein G-Sepharose chromatography. Briefly, the supernatant from cultured hybridoma clones is loaded onto a Protein G-Sepharose column (SIGMA, St. Louis, Mo.) in loading buffer, washed 3 times and the IgG is eluted with elution buffer. These antibodies are then used for screening to evaluate D₁ and D₅ dopamine receptors activation (agonism) potential. This is accomplished using the methodology as outlined in Example 3. Those human monoclonal antibodies which demonstrate agonist activity toward the D₁ and D₅ dopamine receptors are designated candidate compounds.

### Example 7

### Determination of Absolute Force Measurement of a Muscle

The extensor digitorum longus (EDL) and soleus muscles are removed, tendon-to-tendon from the casted mouse leg. A silk suture is tied to each tendon of the isolated muscles and the muscles are placed into a plexiglass chamber filled with Ringer solution (137 mM sodium chloride, 24 mM sodium bicarbonate, 11 mM glucose, 5 mM potassium chloride, 1 mM magnesium sulfate, 1 mM sodium phosphate, 0.025 mM tubocurarine, all at pH 7.4 and oxygenated with 95% oxygen/5% carbon dioxide) constantly bubbled with 95% oxygen/5% carbon dioxide maintained at 25° C. Muscles are aligned horizontally between a servomotor lever arm (Model 305B-LR Cambridge Technology Inc., Watertown Mass., USA) and the stainless steel hook of a force transducer (Model BG-50; Kulite Semiconductor Products Inc., Leonia, N.J., USA) and field stimulated by pulses transmitted between two platinum electrodes placed longitudinally on either side of the muscle. Square wave pulses (0.2 ms duration) generated by a personal computer with a Labview board (Model PCI-MIO 16E-4), Labview Inc., Austin, Tex., USA) are amplified (Acurus power amplifier model A25, Dobbs Ferry, N.Y., USA) to increase titanic contraction. Stimulation voltage and muscle length (Lo) are adjusted to obtain maximum isometric twitch force. Maximum titanic force production (Po) is determined from the plateau of the frequency-force relationship.

### Example 8

### Therapeutic Treatment of Skeletal Muscle Atrophy Using a Human Antibody that is an Agonist of the D₁ and D₅ Dopamine Receptor

A human male subject weighing 50 kg and having significant muscular atrophy of the arms and legs due to prolonged bed rest is treated to reverse the skeletal muscle atrophy. Once each week for a period of 3 months, 15 ml of an aqueous solution of pH 6 comprising an activating antibody of the D₁ and D₅ dopamine receptors are administered to the subject via intravenous injection. The solution comprises the following:

| | Concentration |
|---|---|
| Component | (mg/ml) |
| dopamine receptors activating | 20 |
| antibody | |
| L-histidine HCl | 0.47 |
| L-histidine | 0.3 |
| α, α-trehalose dihydrate | 20 |
| Polysorbate 20 | 0.1 |
| Bacteriostatic Sterile water | qs to 1 ml |

At the end of the treatment period, the subject exhibits measurable increases of muscle mass, strength and mobility of the arms and legs.

### Example 9

### Prophylactic Treatment of Skeletal Muscle Atrophy Using a Human Antibody that is an Agonist of the D₁ and D₅ Dopamine Receptors

A human female subject weighing 55 kg is scheduled for hip joint replacement surgery in one month. The subject is treated to enhance skeletal muscle mass prior to and following surgery to ultimately reduce the level of skeletal muscle atrophy due to muscle disuse during post-surgery recovery. Specifically, once each week for a period of 1 month prior to surgery and for 2 months post-surgery, 18 ml of an aqueous solution of pH 6.0 comprising an activating antibody of the D₁ and D₅ dopamine receptors, are administered to the subject via intravenous injection. The solution comprises the following:

| | Concentration |
|---|---|
| Component | (mg/ml) |
| dopamine receptors activating | 20 |
| antibody | |
| L-histidine HCl | 0.47 |
| L-histidine | 0.3 |
| α, α-trehalose dihydrate | 20 |
| Polysorbate 20 | 0.1 |
| Bacteriostatic Sterile water | qs to 1 ml |

At the end of the treatment period, the subject exhibits measurable preservation of muscle mass, strength and mobility of the arms and legs as compared to the subject's expected status without antibody therapy.

### Example 10

### Prophylactic Treatment of Skeletal Muscle Atrophy Using a Human Antibody that is an Agonist of the D₁ and D₅ Dopamine Receptors

A human female subject weighing 45 kg undergoes a casting procedure to treat a simple fracture of the humerus after a fall. The subject is treated to prevent atrophy of the skeletal muscle of the affected arm and shoulder due to disuse and limited use during fracture healing. Specifically, once each week starting on the day of casting, 13 ml of pH 6.0 comprising the anti-D₁ and D₅ dopamine receptors activating antibody is administered to the subject via intravenous injection. The solution comprises the following:

| | Concentration |
|---|---|
| Component | (mg/ml) |
| dopamine receptor activating | 20 |
| antibody | |
| L-histidine HCl | 0.47 |
| L-histidine | 0.3 |
| α, α-trehalose dihydrate | 20 |
| Polysorbate 20 | 0.1 |
| Bacteriostatic Sterile water | qs to 1 ml |

At the end of the treatment period, the subject exhibits measurable preservation of muscle mass, strength and mobility of the affected arm and shoulder and a reduced course of physical therapy as compared to the subject's expected status and follow-up treatment without antibody therapy.

### Example 11

### Prophylactic Treatment of Skeletal Muscle Atrophy Using Fenoldopam

A human female subject weighing 60 kg is admitted to the hospital in a comatose state. The subject is treated by this method to prevent atrophy of the skeletal muscle of the entire body due to disuse in the comatose state. Specifically, once each day while in the coma, the subject is administered, via slow intravenous infusion, approximately 500 ml of an aqueous solution that is prepared by addition of 5 ml of the following stock solution to 500 ml of sterile saline:

| | Concentration |
|---|---|
| Component | (mg/ml) |
| Fenoldopam | 12 |
| Sodium phosphate | 140 |
| buffer, pH 7.4 | |

As a result of treatment, the subject exhibits measurable preservation of skeletal muscle mass and function, and reduced physical therapy needs during the coma and after regaining consciousness, as compared to the subject's status without drug therapy.

### Example 12

### Therapeutic Treatment of a Patient with Duchenne Muscular Dystrophy Using Fenoldopam

A male subject weighing 40 kg with an existing diagnosis of Duchenne's Muscular Dystrophy is treated with a sustained-release, depot formulation of Fenoldopam in order to improve or retain muscle strength and function over the progression of the disease. Specifically, once each month the subject is administered, via intramuscular injection, 3 ml of an aqueous solution of pH 6.0 comprising the following:

| | Concentration |
|---|---|
| Component | (mg/ml) |
| Fenoldopam | 4 |
| D,L lactic and glycolic | 5 |
| acid copolymer | |

As a result of the treatment, the subject experiences either an improvement or an attenuation of the decline of muscle strength or muscle function in timed-function evaluations as compared to that exhibited during the natural progression of the disease.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for identifying candidate compounds for regulating skeletal muscle mass or function, comprising:
a. contacting a test compound with a D₁ dopamine receptor;
b. determining whether the test compound binds to or activates the D₁ dopamine receptor;
c. selecting those test compounds that bind to or activate the D₁ dopamine receptor, and further determining whether those test compounds regulate muscle mass or function in a skeletal muscle atrophy model system; and
d. identifying those test compounds that regulate muscle mass or function in a skeletal muscle atrophy model system as candidate compounds for regulating skeletal muscle mass or function.

2. A method for identifying candidate therapeutic compounds from a group of one or more candidate compounds which have been previously determined to bind to or activate a D₁ dopamine receptor, comprising:
a. administering the candidate compound to a non-human animal;
b. determining whether the candidate compound regulates skeletal muscle mass or function in the non-human animal; and
c. identifying those candidate compounds that regulate skeletal muscle mass or function in a non-human animal as candidate therapeutic compounds for regulating skeletal muscle mass or function.

3. A method according to claim 1, wherein the D₁ dopamine receptor is expressed on a cell.

4. A method according to claim 3, wherein the D₁ dopamine receptor is expressed on a eukaryotic cell.

5. A method according to claim 1, wherein the amino acid sequence of the D₁ dopamine receptor is greater than 80% identical to the amino acid sequence of SEQ ID NO: 2.

6. A method according to claim 5, wherein the amino acid sequence of the D₁ dopamine receptor is greater than 90% identical to the amino acid sequence of SEQ ID NO: 2.

7. A method according to claim 1, wherein the amino acid sequence of the D₁ dopamine receptor is selected from the group consisting of SEQ ID NOs: 2, 4, 6, 16, 20, 22, 26, 28, 30, and 32.

8. A method according to claim 4, wherein determining whether the test compound activates the D₁ dopamine receptor involves measuring the cellular cAMP level.

9. A method according to claim 8, wherein the cell further comprises a reporter gene operatively associated with a cAMP responsive element and measuring the cellular cAMP level involves measuring expression of the reporter gene.

10. A method according to claim 9, in which the reporter gene is alkaline phosphatase, chloramphenicol acetyltransferase, luciferase, glucuronide synthetase, growth hormone, placental alkaline phosphatase, or a fluorescent protein.

11. A pharmaceutical composition, comprising:
a. a safe and effective amount of a D₁ dopamine receptor agonist; and
b. a pharmaceutically acceptable carrier.

12. A pharmaceutical composition according to claim 11, wherein the D₁ dopamine receptor agonist is 6-chloro-2, 3, 4, 5-tetrahydro-1-(4-hydroxyphenyl)-[1H]-3-benzazepine-7,8-diol.

13. A pharmaceutical composition according to claim 11, wherein the D₁ dopamine receptor agonist is 6-chloro-7,8-dihydroxyl-1-phenyl-2,3,4,5-tetrahydro-1H-3-benzazepine.

14. A pharmaceutical composition comprising a safe and effective amount of a compound for regulating skeletal muscle mass or function identified by a method according to claim 1.

15. A pharmaceutical composition comprising a safe and effective amount of a compound for regulating skeletal muscle mass or function identified by a method according to claim 4.

16. A method for regulating skeletal muscle mass or function in a subject in which such a regulation is desirable, comprising:
a. identifying a subject in which a regulation in muscle mass or function is desirable; and
b. administering to the subject a safe and effective amount of compound that is a D₁ dopamine receptor agonist.

17. A method according to claim 16, wherein the D₁ dopamine receptor agonist is 6-chloro-2, 3, 4, 5-tetrahydro-1-(4-hydroxyphenyl)-[1H]-3-benzazepine-7,8-diol.

18. A method according to claim 16, wherein the D₁ dopamine receptor agonist is 6-chloro-7,8-dihydroxyl-1-phenyl-2, 3, 4, 5-tetrahydro-1H-3-benzazepine.
